SEQUENCE LISTING (10 sequences)

SEQ ID NO: 1 - protein, 4 aa, Artificial Sequence
  Artificial peptide that can function as a spacer.
GFLG

SEQ ID NO: 2 - protein, 4 aa, Artificial Sequence
  Artificial peptide sequence that can function as a spacer.
GLFG

SEQ ID NO: 3 - protein, 13 aa, Artificial Sequence
  VARIANT 3, 4, 11, 13: Xaa = is defined amino acid
MVXXLSNPSRXLX

SEQ ID NO: 4 - protein, 12 aa, Artificial Sequence
  Artificial peptide sequence that binds the HER-2 receptor.
MVKDLSNPSRYL

SEQ ID NO: 5 - protein, 12 aa, Artificial Sequence
  Artificial peptide sequence that binds the HER-2 receptor.
MVRDLSNPSRYL

SEQ ID NO: 6 - protein, 12 aa, Artificial Sequence
  Artificial peptide sequence that binds the HER-2 receptor.
MVKNLSNPSRYL

SEQ ID NO: 7 - protein, 12 aa, Artificial Sequence
  Artificial peptide sequence that binds the HER-2 receptor.
MVKRLSNPSRYL

SEQ ID NO: 8 - protein, 13 aa, Artificial Sequence
  Artificial peptide sequence that binds the HER-2 receptor.
MVRNLSNPSRFLG

SEQ ID NO: 9 - protein, 10 aa, Artificial Sequence
  Artificial peptide sequence that binds the HER-2 receptor.
MVRDLSDPSR

SEQ ID NO: 10 - protein, 3 aa, Artificial Sequence
  Ligand that can bind integrins.
RGD

(19) United States
(12) Patent Application Publication
(10) Pub. No.: US 2003/0124742 A1
(43) Pub. Date: Jul. 3, 2003

(54) Conjugates targeted to target receptors

(76) Inventor: Ramesh K. Prakash, Salt Lake City, UT (US)

Correspondence Address: BINGHAM, MCCUTCHEN LLP, THREE EMBARCADERO, SUITE 1800, SAN FRANCISCO, CA, 94111-4067, US

(21) Appl. No.: 10/226,704

(22) Filed: Aug. 22, 2002

Related U.S. Application Data
(60) Provisional application No. 60/314,282, filed on Aug. 22, 2001.

(51) Int. Cl. G01N 33/543; C08G 63/48; C08G 63/91

(52) U.S. Cl. 436/518; 525/54.1

(57) ABSTRACT

The present invention provides conjugates that preferentially bind to or otherwise associate with specific target cells and methods for using such compositions. Specifically, the present invention provides conjugates comprising a pendant polyalkylene glycol, and a ligand comprising a peptide that preferentially binds to or otherwise associates with a target-receptor. The present invention also provides methods for detecting a disease using such conjugates. In addition, the present invention provides methods for delivering chemical agents and drugs to a mammal using the conjugates of the invention.

### CROSS-REFERENCE TO RELATED APPLICATIONS

The subject invention claims priority to U.S. Provisional Application Serial No. 60/314,282, filed Aug. 22, 2001, the content of which is hereby incorporated by reference into the present disclosure.

### FIELD OF THE INVENTION

The present invention relates generally to compositions that preferentially bind to specific target receptors and methods for using such compositions.

### BACKGROUND OF THE INVENTION

Therapeutic agents that target cell surface receptors or antigens on tumor cells have attracted considerable attention for the treatment of cancer. For example, antigens present on B-lineage cancer cells, such as CD20, are targeted with the anti-CD20 antibody Rituxan® (IDEC Pharmaceuticals Corp. and Genentech, Inc.). IL-4 also is used to target B-cells. Cancer cells expressing epidermal growth factor (“EGF”) or insulin-like growth factor (“IGF”) receptors also are targeted with a binding ligand. Other such ligand-receptor binding pairs are known in the scientific literature for specific cancers. (See, e.g., U.S. Pat. No. 6,025,328 to Pastan and Fitzgerald, Science 254: 1173 (1991); Anderson et al., U.S. Pat. Nos. 5,169,933 and 5,135,736; Thorpe et al., U.S. Pat. No. 5,165,923; Jansen et al., U.S. Pat. No. 4,906,469; Frankel, U.S. Pat. No. 4,962,188; Uhr et al., U.S. Pat. No. 4,792,447; and Masuho et al., U.S. Pat. Nos. 4,450,154 and 4,350,626.)

In general, these therapeutic agents include a cell-targeting moiety, such as a growth factor or an antigen-binding protein, linked to a plant or bacterial toxin. The cell-targeting moiety refers broadly to compounds which serve to deliver therapeutic agents to a specific site for the desired activity. Targeting moieties include, for example, molecules which specifically bind cell surface receptors, such as polyclonal and monoclonal antibodies, cytokines, including interleukins, and factors such as epidermal growth factor (EGF), and the like, are also specific targeting moieties known to bind cell surface receptors. It has also been shown that certain peptides bind to specific tumor-associated antigens in a manner analogous to the binding of antibodies to such antigens. (See e.g., Arap et al., Science 279: 377-380 (1998)).

The uptake of drugs by cells is modulated by absorptive endocytosis at concentrations of drugs clinically achievable in the vicinity of cancer cells. The effectiveness of this pathway depends on the affinity of the polymer-drug conjugate to the cell surface, specifically with the lipid matrix of the plasma membrane. Such interactions could be cancer cell-specific for some particular drug. For example, a differential interaction of doxorubicin with cardiolipin-containing membranes may confer specificity for the drug towards malignant cells (Duarte-Karim et al., Biochem. Biophys. Res. Commun., 71: 658:663 (1976); Tritton et al., Biochem. Biophys. Res. Commun. 84:802-808 (1978)). Cardiolipin, present in the mitochondrial membrane in normal cells, occurs in the plasma membrane of malignant cells (Wallach, D. F. H. (1975) Membrane Molecules Biology of Neoplastic Cells, Elsevier, New York, N.Y.). A low content of cholesterol increases interactions of doxorubicin with the lipid matrix (Hernandez et al., Bioconjug. Chem. 2:398-402 (1991); Gaber et al., Biophys. Chem.70:223-9 (1998)). On the other hand, most cancer cells have a lower content of cholesterol than normal cells (Wallach, D. F. H. (1975) Membrane Molecules Biology of Neoplastic Cells, Elsevier, New York, N.Y.).

Kopecek et al., U.S. Pat. No. 5,258,453 describe compositions for the treatment of cancer tissues comprising a copolymeric carrier and anticancer drug attached to a degradable side chain. (See also Putnam and Kopecek, Adv. Polymer Sci, 122: 550123 (1995)). HPMA (N-(2-hydroxypropyl)methacrylamide) copolymer-adriamycin conjugates comprising a degradable GFLG spacer have also been shown to have anticancer activity. (Omelyanenko et al., Int. J. Cancer, 75:600-608 (1998); and Vasey et al., Clinical Cancer Research, 5: 83-94 (1999)).

However, there remains a need for additional compositions that preferentially target receptors, such as on T lymphocytes, and any receptors involved in developing cancer or antitumor activity. For example, targeting of T lymphocytes would enable therapeutic applications for T-cell-associated diseases and tissue graft rejection. Such T-cell-associated diseases include arthritis, T-cell lymphoma, skin cancers, psoriasis, and diseases resulting from HIV infection.

This invention satisfies these needs and provides related advantages as well.

### DISCLOSURE OF THE INVENTION

It is an object of the present invention to provide compositions and methods for preferentially targeting receptors on specific cell types. In one aspect, the invention provides a conjugate comprising a) a water-soluble biocompatible polymer, b) at least one spacer peptide comprising at least one molecule of a chemical agent releasably coupled to the spacer peptide, and c) at least one targeting peptide linked to the polymer directly or indirectly through a spacer peptide.

In an alternative aspect, the conjugate can comprise (a) a water-soluble, biocompatible polymer, (b) at least one molecule of a chemical agent, which can be releasably and directly coupled to the polymer or indirectly coupled to the polymer through a spacer peptide, and (c) at least one copy of a targeting peptide comprising the sequence “RGD,” for example selected from the group consisting of SEQ ID Nos. 3 through 10 inclusive, directly linked to the polymer or indirectly linked to the polymer through a spacer. The conjugate can have a formula selected from the group consisting of P-[Tₐ-L-S-A]_{c} and [A-S]_{d}-P-[Tₐ-L]_{c}, wherein L is the ligand; A is the chemical agent; S and T are spacers, wherein at least S and optionally T are biodegradable and S and T can be the same or different; P is a water soluble polymer having functional groups compatible with forming covalent bonds with the ligand or a spacer; a is 0 or 1; and c and d are integers of at least 1.0, where the groups bound to P represent one or more

Any water-soluble biocompatible polymer can be used. Exemplary water-soluble biocompatible polymers include but are not limited to polyalkylene oxides, e.g. polyethylene oxide. Suitable polyalkylene oxides include a member selected from the group consisting of polyethylene oxides, alpha-substituted polyalkylene oxide derivatives, polyethylene glycol homopolymers and derivatives thereof, polypropylene glycol homopolymers and derivatives thereof, alkyl-capped polyethylene oxides, bis-polyethylene oxides, copolymers of poly(alkylene oxides), branched polyethylene glycols, star polyethylene glycols, pendant polyethylene glycols, block copolymers of poly(alkylene oxides) and activated derivatives thereof. In a further aspect, the polyalkylene oxide is an alkyl blocked pendant polyethylene glycol (“pPEG”) or mono-methyl blocked pendant polyethylene glycol (“mpPEG”).

The use of alkyl blocked pendant polyalkylene glycol, such as an alkyl blocked pendant polyethylene glycol is advantageous. The multiple pendant groups on the polymer permit the attachment of plural chemical agents to the conjugate, to improve efficacy of the conjugate. For example, the polymer may include 2, 3, 4, 5, 6, 7, 8, or 9 or more molecules of the chemical agent. In one aspect, the polymer includes at least 3, at least 4, at least 5 or at least 6 molecules of the chemical agent.

Additionally, although the use of acyl blocked pendant polyalkylene glycols has similar advantages to the use of alkyl blocked pendant polyalkylene glycols, the use of a diacyl blocked pendant polyalkylene glycol, such as bis-hemisuccinyl pendant polyethylene glycol or monomethyl-hemisuccinyl pendant polyethylene glycol offers the advantage that additional reactive carboxyl group(s) are introduced which can be further derivatized. This is a particular advantage when the pendant groups contain carboxyl moieties, since the possibility of differential reactivity between the hemisuccinyl carboxyl groups and the pendant carboxyl groups is created.

Targeting peptides can be directly linked to the polymer or indirectly linked through a spacer. Examples of such include, but are not limited to, a peptide comprising the sequence GFLG, a peptide comprising the sequence GLFG as well as a peptide comprising a target-receptor-binding peptide.

In a further aspect, the targeting peptide further includes a spacer molecule. Spacer molecules are preferably biodegradable such that the chemical agent is detached from the conjugate by hydrolysis and/or enzymatic cleavage or otherwise in vivo. Once detached, the chemical agent diffuses or is transported to a location within the cell where it can exert its functional effect in vivo. Spacers include, for example, the peptides GFLG (SEQ ID NO: 1) or GLFG (SEQ ID NO: 2) or peptide sequences comprising this linear arrangement of four amino acids. Additional examples are known in the art. (See, e.g., U.S. Pat. No. 6,251,866 to Prakash et al.)

Peptides comprising the sequences GFLG or GLFG have unexpectedly been shown to function as targeting peptides for the delivery of therapeutic agents to cancer cells even though they are not specifically directed to a known cell surface receptor. Thus, for the purposes of this invention, peptides comprising GFLG or GLFG can function as spacer peptides and targeting peptides.

Target-receptor-binding peptides, or alternatively ligands, specifically recognize and bind target or cell surface receptors selectively expressed on certain cell types. In one aspect, the cell surface receptor is specifically or dominantly expressed by a cancer cell, e.g., HER-2, EGF receptors, and transferrin receptors. In an alternative aspect, the target-receptor-binding peptide is directly linked to the polymer at one terminus and linked to a chemical agent at the other. In a further aspect, a spacer peptide is directly linked to the polymer at one terminus and linked to a target-receptor-binding peptide at the other. In a yet further aspect, the target-receptor-binding peptide is linked to the spacer peptide at one terminus and a chemical agent at the other. Chemical agents are linked to any of a spacer, a targeting peptide or a target-receptor-binding peptide so that the chemical agent coupled thereto is released in vivo.

Suitable target-receptor-binding peptides include the peptide having the amino acid sequences selected from the group identified as SEQ ID NOS: 3 through 10, inclusive, and biologically functional equivalents thereof. Such functional equivalents retain functionality in eliciting the biological response. For example, without wishing to be bound to any theory, such biologic response may be achieved by binding the target receptor, even though such binding may not be needed in all cases. However, there may be truncations, deletion variants, chemical modifications, substitution variants, or additional amino acid residues attached to the target-binding receptor peptides.

The target-receptor-binding peptides can have any size, for example, 1000-2000 amino acids or more, or about 1-100 amino acids, about 6-20 amino acid residues, about 6-12 amino acid residues, or about 6-8 amino acid residues.

Changes may be made in the structure of the target receptor-binding peptide while maintaining the desirable functional characteristics. For example, certain amino acid residues may be substituted for other amino acid residues in a protein structure without appreciable loss of interactive binding capacity with the binding site (e.g., antigen binding regions of antibodies or ligand receptor binding sites. Since it is the interactive capacity and nature of a protein that defines that protein's biological functional activity, certain amino acid sequence substitutions can be made in a protein sequence and nevertheless obtain a protein with like properties. It is thus contemplated that various changes may be made in the sequence of an target receptor-binding peptide without appreciable loss of its biological utility or activity.

The conjugates of this invention are particularly useful to deliver chemical agents or drugs in vitro or in vivo to cells bearing a receptor that is recognized by the targeting peptide or the receptor-receptor-binding peptide. Suitable chemical agents include but are not limited to cytotoxins, immunosuppressants, transforming nucleic acids, gene regulators, labels, antigens, and drugs. The chemical agent can also comprise a detectable label.

When the polymer is composed of pendant PEG, multiple peptides can be individually and separately directly linked to the polymer. The spacers may be linked at the other terminus to any one or more of a chemical agent, target peptide or a target-receptor-binding peptide.

Alternatively, the conjugate can comprise multiple target-receptor-binding peptides and/or targeting peptides that are separately and independently directly or indirectly linked to the polymer at one terminus. Chemical agents may or may not be coupled to these peptides. In a further aspect, the conjugate further comprises a detectable label.

The conjugates can be combined with a carrier such as a pharmaceutically acceptable carrier. The agents and compositions of the present invention can be used in the manufacture of medicaments and for the treatment of humans and other animals by administration in accordance with conventional procedures, such as an active ingredient in pharmaceutical compositions. In vitro and in vivo methods using the conjugates of the invention are provided herein.

Methods of delivering a chemical agent to a target-receptor-bearing cell in a population of cells are provided by contacting the population of cells with an effective amount of a conjugate or peptide as disclosed herein. The methods further comprise conditions wherein the ligand or peptide binds to a target receptor on the target-receptor-bearing cells. Methods of detecting a disease associated with elevated levels of soluble target receptor in circulation also are provided by mixing or contacting a conjugate or peptide as disclosed herein with a body fluid to be tested under conditions suitable for forming a complex of the conjugate or peptide and the soluble target receptor on the target-receptor in said body fluid and determining whether said complex is present at elevated levels as compared to normal individuals. Suitable examples of body fluids include, but are not limited to blood, tissue, saliva and urine.

### BRIEF DESCRIPTION OF THE DRAWINGS

FIG. 1 is a graph showing WL38 (also identified as TT38) binding to HER2 Receptor.

FIG. 2 shows the results of the binding study of PEGTyrWL68DXR conjugates to cell surfaces.

FIG. 3 shows the in vitro cytotoxicity activity of doxorubicin conjugates against human T lymphoma cell line HUT 78.

FIG. 4 is a graph showing the in vitro cytotoxicity activity of doxorubicin conjugates against human ovarian cancer cell line A-2708R.

FIG. 5 is a graph showing the efficacy of doxorubicin conjugates in vivo in a murine model of human cutaneous T cell lymphoma.

FIG. 6 is a graph showing tumor growth inhibition by doxorubicin conjugates in a murine tumor model of human ovarian cancer cell line.

FIG. 7 is a typical HPLC chromatogram of the organic phase extraction of the heart tissue of mice administered with PEG-WL00-DXR conjugates.

FIG. 8 is a graph showing the body distribution of DXR administered as TM 1192-029 conjugate. The data were obtained 72 hours after the administration of 2.5 mg/kg of DXR equivalent.

FIG. 9 is a graph showing the correlation between the IC₅₀ of a composition according to the present invention and the IC₅₀ of free doxorubicin in HER-2 negative fresh breast carcinoma specimens.

FIG. 10 is a graph showing the correlation between the IC₅₀ of a composition according to the present invention and the IC₅₀ of free doxorubicin in HER-2 positive fresh breast carcinoma specimens.

### MODES FOR CARRYING OUT THE INVENTION

Throughout this disclosure, various publications, book chapters, books, manuals, patents and published patent specifications are referenced by an identifying citation. The disclosures of these publications, patents and published patent specifications are hereby incorporated by reference into the present disclosure to more fully describe the state of the art to which this invention pertains.

Definitions

The practice of the present invention will employ, unless otherwise indicated, conventional techniques of immunology, molecular biology, microbiology, cell biology and recombinant DNA. These methods are described in the following publications. See, e.g., Sambrook, et al. MOLECULAR CLONING: A LABORATORY MANUAL, 2^{nd} edition (1989); CURRENT PROTOCOLS IN MOLECULAR BIOLOGY (F. M. Ausubel, et al. eds., (1987)); the series METHODS IN ENZYMOLOGY (Academic Press, Inc.); “PCR: A PRACTICAL APPROACH” (M. MacPherson, et al., IRL Press at Oxford University Press (1991)); PCR 2: A PRACTICAL APPROACH (M. J. MacPherson, B. D. Hames and G. R. Taylor eds. (1995)); ANTIBODIES, A LABORATORY MANUAL (Harlow and Lane, eds. (1988)); and ANIMAL CELL CULTURE (R. I. Freshney, ed. (1987)).

As used herein, the singular forms “a,” “an,” and “the” include plural references unless the context clearly dictates otherwise. Thus, for example, a composition containing “a ligand” includes reference to two or more ligands. Similarly, a “chemical agent” includes reference to one or more of such chemical agents that may be the same or different chemical agents, and reference to “a spacer” includes reference to two or more spacers.

As used herein, the term “comprising” is intended to mean that the compositions and methods include the recited elements, but not excluding others. “Consisting essentially of” when used to define compositions and methods, shall mean excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this invention. Embodiments defined by each of these transition terms are within the scope of this invention.

As used herein, “peptide” means peptides of any length and includes proteins. The terms “polypeptide” and “oligopeptide” are used herein without any particular intended size limitation, unless a particular size is otherwise stated.

As used herein, the terms “target-receptor-binding peptide,” “peptide preferentially binding to a target receptor” or “peptide specifically binding to a target receptor” or “ligand” refer to a peptide capable of binding to a target receptor on a cell.

As used herein, the term “target receptor” refers to any moiety on the cell surface to which the target receptor binding peptide binds or otherwise associates with. In one aspect, the binding to the receptor facilitates internalization of the conjugate into the cell. Internalization into the cell can occur by any mechanism including passive diffusion and endocytosis. In one embodiment, the target receptor is a receptor that promotes endocytosis of the target receptor binding peptide upon binding of the target receptor binding peptide to the target receptor.

As used herein, the term “RGD” refers to the tripeptide Arg-Gly-Asp (SEQ ID No. 10). As used herein, the term “RGD receptor” refers to a receptor that binds to the RGD motif. RGD is known to bind to integrins.

As used herein, the terms “HER-2” or “HER-2 receptor” (also known as c-erbB-2 or neu) refer to the human epidermal growth factor receptor-2.

As used herein, the terms “biologically functional equivalents” or “chemically modified equivalents” refer to compositions wherein one or more amino acids of the peptides have been chemically modified, or substituted by its analogues without a significant loss of its target receptor binding/associating activity. Various types of chemically modified amino acids and analogues are commercially available and are well known to one skilled in the art.

Amino acid substitutions are generally based on the relative similarity of the amino acid side-chains relative to, for example, their hydrophobicity, hydrophilicity, charge, size, and the like. Based upon these considerations, the following conservative substitution groups or biologically functional equivalents have been defined: (a) Cys; (b) Phe, Trp, Tyr; (c) Gln, Glu, Asn, Asp; (d) His, Lys, Arg; (e) Ala, Gly, Pro, Ser, Thr; and (f) Met, Ile, Leu, Val. (Dayhoff et al., Atlas of Protein Sequence and Structure (Nat'l Biomed. Res. Found., Washington, D.C., 1978), hereby incorporated by reference).,

To effect more quantitative changes, the hydropathic index of amino acids may be considered. Each amino acid has been assigned a hydropathic index on the basis of its hydrophobicity and charge characteristics, which are as follows: isoleucine (+4.5); valine (+4.2); leucine (+3.8); phenylalanine (+2.8); cysteine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is generally understood in the art. J. Kyte & R. Doolittle, 157 J. Mol. Biol. 105-132 (1982), incorporated herein by reference. It is known that certain amino acids may be substituted for other amino acids having a similar hydropathic index or score and still retain a similar biological activity. In making changes based on the hydropathic index, the substitution of amino acids whose hydropathic indices are within ±2 is preferred, within ±1 is particularly preferred, and within ±0.5 is even more particularly preferred.

It is also understood that an amino acid can be substituted for another having a similar hydrophilicity value and still obtain a biologically equivalent protein. As detailed in U.S. Pat. No. 4,554,101, the following hydrophilicity values have been assigned to amino acid residues: arginine (+3.0); lysine (+3.0); aspartate (+3.0); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). In making changes based upon similar hydrophilicity values, the substitution of amino acids whose hydrophilicity values are within ±2 is preferred, within ±1 is particularly preferred, and within ±0.5 is even more particularly preferred.

As used herein, the terms “normal subject” or “normal control” refer to a subject who does not suffer from the particular condition being tested for (e.g., cancer).

As used herein, the term “macromolecule” refers to a composition comprising a water soluble polymer with a ligand and a chemical agent releasably coupled thereto. Preferably the polymer is a polyalkylene oxide and the ligand is an oligopeptide. The chemical agent can be from many different classes of molecules, as explained in more detail herein.

As used herein, the terms “releasably coupled” or “releasable, covalent bond” or “covalently, releasably coupled” refer to covalent bonds of the ligand, the chemical agent and the biocompatible, biodegradable polymer. In particular, a conjugate comprising a chemical agent “covalently, releasably coupled to the polymer” refers to the embodiment wherein the chemical agent is covalently bonded to a component of the conjugate, but is releasable under suitable conditions, e.g., by enzymatic or pH-related cleavage of the covalent bond, e.g., by receptor-mediated endocytosis of the conjugate into the target cell. The chemical agent may be releasable by being attached to a portion of the conjugate, such as the polymer, via a degradable linkage (e.g., a peptide linkage that degrades in the presence of a protease).

As used herein, the term “prodrug” refers to a chemical agent that is chemically modified to overcome a biological barrier. When a chemical agent is converted into its prodrug form, its biological activity is eliminated or substantially reduced, but the biological barrier that inhibited its effectiveness is no longer problematic. The chemical group that is attached to the chemical agent to form the prodrug, i.e. the “pro-moiety”, is removed from the prodrug by enzymatic or nonenzymatic means to release the active form of the chemical agent. See, Albert, Nature 182:421 (1958). The instant compositions are prodrugs because the chemical agent that has the selected effect when internalized in receptor-bearing cells is modified with a targeting peptide, a water soluble polymer, and, optionally, spacers such that the composition is delivered to the target receptor and/or target-receptor-bearing cells, thus penetrating the cell membrane thereof The biological effect of the chemical agent is greatly reduced or eliminated until the composition is delivered intracellularly and the chemical agent is released from the remainder of the composition by biodegradation of the spacer.

As used herein, the term “chemical agent” refers to any substance that has a selected effect either in vivo or in vitro. Examples of biological effects include a cytotoxic effect or an effect on gene regulation. Examples of chemical agents include a transforming nucleic acid, a gene regulator, a label, a drug, and a polypeptide. Thus, chemical agents include cytotoxins, gene regulators, nucleic acids, labels, antigens, drugs, and the like. A “transforming nucleic acid” (RNA or DNA) can be replicated and/or expressed by a cell. A “gene regulator” is a nucleic acid that interacts with regulatory sequences or regulatory factors in the cell to influence gene expression in a selected manner. A detectable “label” allows identification of cells that have interacted with the compositions of the present invention by detection of the label. “Drugs” or “pharmacologically active compounds” can be used to ameliorate pathogenic effects or other types of disorders. Particularly useful chemical agents include polypeptides. Some chemical agents are active fragments of biologically active proteins, or are specific antigenic fragments (e.g., epitopes) of antigenic proteins.

As used herein, the terms “drug” or “pharmacologically active agent” refer to any chemical material or compound suitable for administration to a mammal that stimulates a desired biological or pharmacological effect in such mammal. Examples include cytotoxins and immunosuppressant drugs. Examples of cytotoxins include doxorubicin (“DXR”), taxol, cisplatin, methotrexate, cyclophosphamide, and derivatives of any thereof. Doxorubicin is available commercially, for example from Sigma, St. Louis, Mo. Other drugs include Daunorubicin, Epirubicin, Idarubicin, Mitoxantrone, Novantrone, Actinomycin D and Amsacrine. Examples of immunosuppressants include cyclosporin, rapamycin, and FK506. Examples of anticancer drugs include aminopterin, folic acid, 10-ethyldeazaaminopterin, trimetrexate, piritrexim, tomudex, Daraprim, lemotrexol, fluorouracil, 5-azacytidine, 2′,2′-difluoro-2′ deoxycytidine, brequinar, pyrazofurin, 6-azauridine, 5-ethynyluracil, allopurinol, acivicin, leucovorin, acyclovir, ganciclovir, and derivatives thereof. Other anticancer drugs are described in Cancer Medicine, Hollan, J. F., ed., (1997).

As used herein, the term “carrier” refers to any carrier, such as water soluble polymers, particulates, or liposomes to which a conjugate according to the present invention can be combined or coupled. Such carriers can, for example, increase the molecular size of the compositions and may provide added selectivity, biodistribution, control over the release rate of the drug from the composition and/or stability. Such selectivity can arise because, for example, carrier-containing compositions are too large to enter cells by passive diffusion, and thus are limited to entering cells through receptor-mediated endocytosis. The potential for use of such carriers for targeted drug delivery has been established. (See, e.g., J. Kopecek, Biomaterials 5:19 (1984); Schacht et al., Polysaccharides as Drug Carriers, in Controlled-Release Technology 188 (P. I. Lee & W. R. Good, eds., 1987); Hudecz et al., J. Controlled Release 19:231 (1992); Brich et al., J. Controlled Release 19:245 (1992). Illustrative water soluble polymers include dextran, inulin, poly(L-lysine) with modified epsilon-amino groups, poly(L-glutamic acid), N-substituted methacrylamide-containing synthetic polymers and copolymers, and the like.

As used herein, the term “effective amount” refers to an amount sufficient to produce a selected effect. For example, a selected effect of a composition containing a cytotoxin as the chemical agent could be to kill a selected proportion of target-receptor-bearing cells within a selected time period. An effective amount of the composition would be the amount that achieves this selected effect, and such an amount can be determined by a person skilled in the art.

As used herein, the term “pendant” as used in reference to a polyalkylene glycol and the like refers to a polymer that includes a plurality of pendant functional groups dispersed along the polymer chain. The pendant functional groups typically comprise, or can be modified to comprise, reactive groups that permit further modification and covalent attachment of other molecules to the polymer. The number of pendant groups on a single polymer can vary, including within a conjugate preparation. For example, pendant groups can provide attachment points for targeting ligands and/or chemical agents. In one embodiment, the polymer molecule includes two to eight pendant groups. In other embodiments, the polymer molecule includes more than eight pendant groups.

As used herein, the terms “alkyl blocked” or “alkyl capped” polyalkylene glycol refers to the form of the polymer when one or more of the terminal hydroxyl groups are capped with an alkyl group, such as a methyl group.

As used herein, the term “mpPEG” refers to mono-methyl blocked pendant polyethylene glycol. The term “pPEG” refers to a pendant polyethylene glycol. The term “dmpPEG” refers to a pendant polyethylene glycol dimethyl ether.

As used herein, the term “chemically conjugating the ligand and the chemical agent to the water soluble polymer” and the like refer to covalently bonding the ligand and chemical agent to each other, preferably by way of a spacer moiety, and conjugating the resulting ligand/agent conjugate to the water soluble polymer.

The present invention relates generally to compositions that preferentially bind or otherwise associate with specific target receptors. The present invention also provides methods for detecting a disease using such conjugates. In addition, the present invention provides methods for delivering chemical agents to cells in vitro or in vivo, using the conjugates of this invention.

I. Conjugates Targeted to Target Receptors

The present invention provides conjugates comprising a water-soluble, biocompatible polymer, at least one peptide spacer and at least one targeting peptide. The invention also provides conjugates comprising a water-soluble, biocompatible polymer, a targeting peptide, and a chemical agent. It is understood by one of ordinary skill in the art that the conjugates of the present invention can comprise various other polymers and ligands and peptides, in addition to the specific embodiments disclosed herein. Furthermore, it is understood by one of ordinary skill in the art that the conjugates of the present invention can target various target receptors, in addition to the specific receptors described herein.

A. Water-Soluble Polymers

Various water-soluble polymers can be used in the compositions and methods of the present invention. (See e.g., Duncan, “Drug-polymer conjugates: potential for improved chemotherapy, Anti-Cancer Drugs 3:175-210 (1992)). The water soluble polymer is preferably a polyalkylene oxide. Within this group of substances are alpha-substituted polyalkylene oxide derivatives, such as methoxypolyethylene glycols or other suitable alkyl-substituted derivatives. Suitable alkyl-substituted derivatives include, but are not limited to, C₁-C₄ alkyl groups.

The polyalkylene oxide is preferably a monomethyl-substituted pendant PEG homopolymer. However, other poly(alkylene oxides) can also be used, including, but not limited to, polyethylene glycol (PEG) homopolymers and derivatives thereof; polypropylene glycol homopolymers and derivatives thereof; alkyl-capped polyethylene oxides; bis-polyethylene oxides; copolymers of poly(alkylene oxides); and block copolymers of poly(alkylene oxides) and activated derivatives thereof. Other preferred PEGs are branched, pendant and star PEGs, such as those commercially available from Shearwater Polymers, Inc. (Huntsville, Ala.). (Gnanou et al., Makromol. Chem. 189:2885 (1988); Rein et al., Acta Polymer 44: 225 (1993); Merrill, U.S. Pat. No. 5,171,264; Poly(Ethylene Glycol) Chemistry in Biotechnical and Biomedical Application, J. Milton Harris, (1992).

The molecular weight of the pendant polymer is not limited to a particular number, but only by the relevant practical considerations such as crowding, handling, physical characteristics (e.g., viscosity, density, solubility, etc.) and the acceptability in a composition for administration to a mammal. For example, in some aspects, the PEG-based polymers have average molecular weights of from about 200 to about 50,000. In another embodiment, PEG-based polymers having average molecular weights of from about 2,000 to about 20,000 are used. PEG is preferred because it is inexpensive, approved by the FDA for administration to humans, and is resistant to eliciting an antibody response. Poly(ethylene oxide) (PEO), which forms a polymer with the same structure as PEG, is another preferred water soluble polymer. One preferred polymer for the present invention comprises a pendant PEG. Another preferred polymer of the present invention comprises a star shaped PEG.

In one aspect, the polymer is a blocked pendant polyalkylene glycol, such as an alkyl blocked pendant polyalkylene glycol. The polymer is preferably an alkyl blocked pendant polyethylene glycol. For example, the alkyl blocked pendant polyethylene glycol can be a mono-methyl blocked pendant polyethylene glycol, or a dimethyl blocked pendant polyethylene glycol.

In yet another aspect, the pendant polymer is a linear polymer comprising terminal hydroxyl groups. At least one of the terminal hydroxyl groups is preferably capped with a nonreactive functional group such as an alkyl group. For example, the polymer can be mono-methyl blocked (i.e., having one terminal hydroxyl capped with a methyl group) or dimethyl blocked with a methyl capping group on two of the terminal hydroxyls.

In the case of a mono-alkyl blocked polyalkylene glycol, the remaining free hydroxyl groups can be further blocked by an acyl group such as acetyl or hemisuccinyl (e.g., via an ester bond from reaction with a mono or dicarboxylic acid or derivative). In the case of hemisuccinyl or other diacyl compound, additional reactive groups (carboxyl groups) can be introduced for further derivatization. Alternatively, a non-alkylated pendant polymer containing two or more terminal hydroxyl groups can be capped with two acyl or diacyl compounds such as acyl or hemisuccinyl to yield a bi-substituted bis-blocked polymer. When hydroxyl groups are capped with bis-hemisuccinyl or other bis-diacyl compound, two additional reactive groups (e.g., carboxyl groups) can be introduced at the ends of each polymer chain for further derivatization.

Blocked pendant polyalkylene glycols can be made using synthetic methods available in the art. Pendant PEGs can also be obtained commercially, such as from Innophase Corporation (Westbrook, Conn.). Alkyl-blocked pendant polyalkylene glycols are generally prepared by alkoxylation of monoalkylalkylene glycols using alkylene oxide and pendant groups introduced by methods available in the art. Monomethyl PEGs are also commercially available. Dialkyl blocked pendant polyalkylene glycols are generally prepared from monoalkyl PEGs by reaction with dialkyl sulfate and a strong base, or via the tosylate ester by reaction with alkoxide and subsequent attachment of pendant groups by methods available in the art (see, for example, Advanced Organic Chemistry, J. March, Wiley: N.Y., Fourth Edition (1992) pp. 386-387). Acyl and diacyl blocked pendant PEGs can be prepared, for example by reaction of activated carboxyl derivatives such as acyl or cyclic anhydrides with the pendant polyalkylene glycols or monoalkyl blocked pendant polyalkylene glycols (See Advanced Organic Chemistry, J. March, Wiley: N.Y., Fourth Edition (1992) pp. 392-396).

The use of alkyl blocked pendant polyalkylene glycol, such as an alkyl blocked pendant polyethylene glycol is advantageous. The multiple pendant groups on the polymer permit the attachment of plural chemical agents to the conjugate, to improve efficacy of the conjugate. For example, the polymer can include from two to nine molecules of the chemical agent. Preferably, the polymer includes from three to six molecules of the chemical agent. In some embodiments, the polymer can include more than nine molecules of the chemical agent.

Although acyl blocked pendant polyalkylene glycols have similar advantages to alkyl blocked pendant polyalkylene glycols, diacyl blocked pendant polyalkylene glycol offers the advantage of introducing additional reactive carboxyl groups that can be further derivatized. This is a particular advantage when the pendant groups contain carboxyl moieties, since the possibility of differential reactivity between the terminal hemisuccinyl carboxyl groups and the pendant carboxyl groups is created. Examples of diacyl blocked pendant polyalkylene glycols include, but are not limited to, bis-hemisuccinyl pendant polyethylene glycol and monomethyl-hemisuccinyl pendant polyethylene glycol.

In another aspect, the polymer may comprise more than one chemical agent which may be the same or different. For example, in one aspect, the polymer may comprise a number of doxorubicin molecules and a number of cyclosporin molecules.

B. Peptide Compositions

In one aspect of the invention, the present invention provides conjugates capable of eliciting a selected effect when delivered to a selected cell type. For example, the conjugate comprises a ligand configured for binding to a target receptor on target-receptor-bearing cells. In one aspect, the binding of the receptor to the ligand stimulates internalization into the cell. The conjugate can stimulate internalization into the cell by any mechanism. For example, the conjugate can stimulate internalization into the cell by receptor-mediated endocytosis. The conjugate can further comprise a chemical agent and a water soluble polymer having functional groups compatible with forming releasable, covalent bonds with the binding ligand.

In one embodiment, the present invention provides a conjugate comprising a water-soluble, biocompatible polymer, and a target-receptor-binding peptide or ligand comprising a peptide having the sequence Arg-Gly-Asp (SEQ ID No. 10), which selectively binds to receptors on cell surfaces, e.g. integrins. In another embodiment, the present invention provides a conjugate comprising a water-soluble, biocompatible polymer, and a target-receptor-binding peptide or ligand comprising a peptide that selectively binds to HER-2 receptors. Ligands that bind to the HER2 receptor are known in the art. Specific examples of peptides that preferentially bind the HER2 receptor comprise a sequence selected from the group consisting of:

MVX₁ X₂LSNPSRX₃LX₄ (SEQ ID No. 3)

wherein X₁ is K or R;

wherein X₂ is K or R;

wherein X₃ is Y or F; and

X₄ is absent or G;

MVKDLSNPSRYL (“WL 38”) (SEQ ID No. 4), MVRDLSNPSRYL (“WL 60”) (SEQ ID No. 5), MVKNLSNPSRYL (“WL 61”) (SEQ ID NO: 6), MVKRLSNPSRYL (“WL 62”) (SEQ ID NO: 7), MVRNLSNPSRFLG (“WL 68”) (SEQ ID NO: 8), MVRDLSDPSR (“WL 71”) (SEQ ID NO: 9), and biologically functional equivalents thereof

In an alternative embodiment, the conjugate comprises at least one copy of a ligand peptide selected from the group consisting of SEQ ID Nos. 3 through 10 inclusive, directly linked to the polymer or indirectly linked to the polymer through a spacer, wherein the conjugate has a formula selected from the group consisting of P--[Tₐ-L-S-A]_{c} and [A-S]_{d}-P-[Tₐ-L]_{c}, wherein L is the ligand; A is the chemical agent; S and T are spacers, wherein at least S is biodegradable and S and T can be the same or different; P is a water soluble polymer having one or more functional groups compatible with forming covalent bonds with the ligand or a spacer; a is 0 or 1; and c and d are integers of at least 1.

The compositions and methods of the present invention are not limited to specific target receptor-binding peptides nor to specific ligands having specific peptide sequences. The compositions of the present invention encompasses conjugates comprising ligands having peptides that selectively bind to or otherwise associate with target cells having any receptors involved in the development of cancer and antitumor activity. Target cells can include, but are not limited to, cancer cells, T cells, HER-2 receptor bearing cells, RGD-receptor bearing cells, e.g., endothelial cells expressing integrins such as fibronectin receptors, EGF-receptor bearing cells, or transferrin-receptor bearing cells, e.g., malignant tumor cells.

In one embodiment, the targeting peptide or ligand comprises peptides which contain a biodegradable spacer such that the chemical agent, if any, is detached from the composition by hydrolysis and/or enzymatic cleavage inside the target cell, especially in lysosomes. Such cleavage may occur outside the target cells. Hydrolysis may be in some aspect pH driven (i.e., acid/base driven). Once the chemical agent is detached it can exert its functional effect in the cell. It should be recognized that in some aspects the chemical agent may remain uncleaved (i.e., not released from the polymer) but it is still able to elicit its biological effect. Illustrative spacers are the peptides Gly-Phe-Leu-Gly (SEQ ID NO:1) and Gly-Leu-Phe-Gly (SEQ. ID. No. 2). Equivalent peptide spacers are well known in the art.

The conjugates of the present invention comprise peptides that can be employed as ligands, spacers, and/or chemical agents. The peptides according to the invention can be made using a variety of techniques known in the art, including, but not limited to, organic synthesis and recombinant DNA methods. (See, Merrifield et al., Biochemistry 21: 5020 (1982); Houghten, Proc. Nat'l Acad. Sci. USA 82: 5131 (1985); Bodanszky & Bodanszky, The Practice of Peptide Synthesis (Springer-Verlag 2d ed., 1994), incorporated herein by reference). Techniques for chemical conjugation of peptides with other molecules are also known in the art.

A fusion protein according to the invention can be made by expression in a suitable host cell of a nucleic acid containing an oligonucleotide encoding a ligand, spacer and/or chemical agent. Such techniques for producing recombinant fusion proteins are well-known in the art. (See e.g., Sambrook et al., Molecular Cloning: A Laboratory Manual (2d ed., 1989), incorporated herein by reference). Reagents useful in applying such techniques, such as restriction endonucleases and the like, are widely known in the art and commercially available from any of several vendors. Peptide portions of the compositions can be produced in a genetically engineered organism, such as E. coli, as a fusion protein. A hybrid gene encoding the fusion protein can be inserted into an organism such that the fusion protein is expressed. The fusion protein can then be purified by standard methods, including affinity chromatography. Peptides containing a ligand, spacer, and/or peptide chemical agent can also be constructed by chemical synthesis. Short peptide ligands are generally preferred because short peptides can be manipulated more readily.

Another aspect of the present invention features a method for specifically effecting a desired activity in a target receptor contained in a heterogeneous population of cells, by contacting a population of cells with a composition or conjugate of this invention. The compositions or conjugates of the invention selectively bind or otherwise associate with cells bearing a target receptor in the mixed population. This application employs, except where otherwise indicated, standard techniques for manipulation of peptides and for manipulation of nucleic acids for expression of peptides. Techniques for conjugation of oligopeptides and oligonucleotides are known in the art. (See Zhu et al., Antisense Res. Dev. 3: 265 (1993); Zhu et al., Proc. Nat'l Acad. Sci. USA 89: 7934 (1992); Rigaudy et al., Cancer Res. 49: 1836 (1989)).

C. Chemical Agents

In some embodiments of the present invention, the compositions are constructed by chemically conjugating the ligand and chemical agent to the water soluble polymer. In particular embodiments, a biodegradable spacer moiety is used to form a linkage between the ligand and the chemical agent. Chemical agents may comprise cytotoxins, including radionuclides, for selective killing or disabling of cells; nucleic acids or peptides for genetically transforming or regulating gene expression in cells; drugs or other pharmacologically active agents including immunosuppressants, for achieving a selected therapeutic effect; labels, including fluorescent, radioactive, and magnetic labels, for permitting detection of cells that have bound and/or taken up the compositions; and the like.

In preferred embodiments, the conjugates according to the present invention further comprise a protease digestion site, such that the chemical agent can be separated from the remainder of the composition in vivo (either within the cell or outside the cell). Such a protease-susceptible biodegradable peptide can be added regardless of whether the peptide portions of the composition are synthesized chemically or as expression peptides in a genetically engineered organism. In the latter case, nucleotides encoding the protease susceptible spacer can be inserted into the hybrid gene encoding the ligand or a peptide chemical agent by techniques well known in the art. In one illustrative embodiment, the protease-susceptible peptide is designed to be cleaved by proteolysis in the lysosome of the target cell. The composition that is internalized, for example, by endocytosis, is packaged in an endocytic vesicle, which is transported to a lysosome. Once in the lysosome, the protease-susceptible portion is cleaved, and the chemical agent is then available to be transported to the cytoplasm.

D. Target Receptors

The compositions of the present invention encompass conjugates comprising target-receptor binding peptides that preferentially bind to or otherwise associate with cell surface markers or receptors, including but not limited to RGD receptors (integrins), HER-2 receptors, EGF receptors, transferrin receptors.

1. RGD

Arg-Gly-Asp (“RGD”, SEQ ID NO: 10) was originally identified as the sequence within fibronectin that mediates cell attachment, and has been found in numerous other proteins. For example, a subset of the integrins, a family of cell-surface proteins that act as receptors for cell adhesion molecules, recognize the RGD motif within their ligands. The binding of RGD in these integrins mediates both cell-substratum and cell-cell interactions.

RGD peptides and mimetics have been proposed as potential therapeutic agents for the treatment of diseases such as thrombosis and cancer. (D'Souza et al., Trends Biochem. Sci. 16: 246-250 (1991)). Tumor growth and its metastases to different organs require new blood vessels for its nourishment. Inhibition of angiogenesis could be a means of retarding tumor growth, and possibly induce its regression. The receptor integrins are induced in angiogenic vasculature and in many human tumors, but are absent or expressed only at low levels in normal endothelial cells of blood vessel. The majority of integrins recognize the sequence Arg-Gly-Asp (“RGD”), an element of integrin recognition site in many different adhesive proteins.

By phage displaying library, cyclic peptide containing RGD has been shown to exhibit potency against breast carcinoma and malignant melanoma when injected intravenously in tumor-bearing nude mice. Present treatments use cyclic peptides containing RGD to inhibit angiogenesis and cyclic peptide RGD coupled to drugs to kill tumor blood vessels. (See e.g., Rouslahti, Ann. Rev. Cell Div. Biol. 12: 697-715 (1996); O'Neil et al., Proteins 14: 509-515; Koivunen et al., J. Biol. Chem. 268: 20205-20210 (1993); Healy et al., Biochemistry 34: 3948-3955 (1995); Koivunen et al., J. Cell. Biol. 124: 373-380 (1994); Koivunen et al., Biotechnology 13: 265-270 (1995); Pasqualini et al., Biotechnology 15: 542-546 (1997); and Arap et al., Science 279: 377-380 (1998)).

Unlike treatments using cyclic peptides containing RGD, the present invention provides compositions comprising conjugates of linear RGD to polymeric drug. Coupling of multiple RGD gives high affinity binding to integrins. Conjugating RGD to PEG also allows longer circulation in the blood. Specifically, the spacer peptide will be stable in blood, but releases the drug to integrin-expressing endothelial cells. Thus, the conjugates of the present invention are effective in tumor targeting and in cancer treatment. The compositions and methods of the present invention utilizing conjugation of RGD to polymeric drug are also simpler and cheaper than currently used methods utilizing cyclic RGD peptides.

2. HER-2 Receptors

Preclinical studies have suggested that HER-2 overexpression enhances metastatic potential of breast cancer cells. In general, HER-2 amplification is found to be associated with more aggressive pathological features. (Sahin, Adv. Anat. Pathol. 7: 158-66 (2000); van de Vijver, Eur. J. Cancer Suppl 1: 1-7 (2001)). HER-2 can also be a prognostic factor and target of therapy in tumors of the gastrointestinal tract. (Ross et al., Cancer Invest. 19: 554-568 (2001)). The proto-oncogene designated erbB2 or HER-2 encodes a 185-kilodalton transmembrane tyrosine kinase (p185^{erbB2}). A 45-kilodalton protein heregulin-α (HRG-α) has been purified from the conditioned medium of a human breast tumor cell line. Scatchard analysis of the binding of recombinant HRG to a breast tumor cell line expressing p185^{erbB2} showed a single high affinity binding site. Heregulin transcripts have been identified in several normal tissues and cancer cell lines. (See e.g., Holmes et al., Science 256: 1205-1209 (1992)). Thus, therapies directed at preventing the function of HER-2 receptors are potentially useful anti-cancer treatments.

3. EGF Receptors

Epidermal growth factor receptors (“EGF receptors”) are expressed at high levels in about one third of epithelial cancers, and autocrine activation of EGF receptors appears to influence the growth of many tumors. Thus, therapies directed at preventing the function of these receptors are potentially useful anti-cancer treatments. (Mendelsohn et al., Oncogene 19: 6550-6555 (2000); Mendelsohn, Endocr. Relat. Cancer 8: 3-9 (2001)).

4. Transferrin Receptors

Transferrin receptors are membrane glycoproteins whose function is to mediate cellular uptake of iron from a plasma glycoprotein, transferrin. Iron uptake from transferrin involves the binding of transferrin to the transferrin receptor, internalization of the transferrin with an endocytic vesicle by receptor-mediated endocytosis, and the release of iron from the protein by a decrease in endosomal pH., Transferrin receptors are highly expressed on immature erythroid cells, placental tissue, and rapidly dividing cells, both normal and malignant. (Ponka et al., Int. J. Biochem. Cell Biol. 31: 1111-1137 (1999). They have also been correlated with the degree of malignancy and therefore have been suggested as a tumor selective target. Hopkins, C. et al. J. Cell Biol. 97:508-521 (1983) and Trowbridge, I. S., et al. Nature 294:171-173 (1981)). Transferrin can therefore be used as a ligand in conjugates targeted towards transferring-receptor bearing cells.

### EXAMPLES

The following examples are intended to illustrate, not limit the invention described herein. In the experimental disclosure which follows, the following abbreviations apply: M (molar); μm or μmole (micromolar); mmol (millimoles); mol (moles); g (grams); mg (milligrams); 1 or L (liters); ml (milliliters); 1l (microliters); ° C. (degrees Centigrade); IPA (isopropyl alcohol); DMF (dimethylformamide); v (volume); TLC (thin layer chromatography); DMAP (4-dimethylaminopyridine); EDC [1-(3-dimethylaminopropyl)-3-ethylcarbodiimide hydrochloride); DXR (doxorubicin); DIEA (diisopropylethylamine); CH₃CN (acetonitrile); MeOH (methanol); PA (propionic acid); SA (succinic acid); TMS (tetramethylsilane); NMR (Nuclear Magnetic Resonance); and SEC (Size Exclusion Chromatography).

### EXAMPLE 1

7.8 g of branched, 8-arm PEG-COOH (20 kDa; Shearwater Polymers, Inc., Huntsville, Ala.) and 0.25 g of p-nitrophenol were dissolved in 500 ml of tetrahydrofuran (THF) (Aldrich), making a 3.6 mM solution with respect to the p-nitrophenol. The solution was cooled in an ice bath, and 0.9341 g of dicyclohexylcarbodiimide (DCC, Sigma) in THF was added to the reaction mixture in four aliquots. The reaction solution was stirred for 45 minutes in the ice bath, and then continued for some time at room temperature. The reaction solution was then filtered through filter paper, and the filtrate was concentrated by evaporating the solvent with a rotary evaporator using a water pump. The clear concentrated solution (30 ml) was added to ether (750 ml). The precipitate was filtered, washed in ether, and dried in air. An aliquot of the product was dissolved in 0.1 N NaOH, and the concentration of the liberated p-nitrophenol was estimated by spectrophotometry at 400 nm using a molar extinction coefficient of ε=1.8×10⁴ l/mol-cm. The product (PEG-ONp), was determined to have an ONp content of 201.3 μmol/g.

PEG-ONp (0.168 g, ONp content 201.3 μmol), prepared as described above, was dissolved in 2 ml anhydrous dimethylformamide (DMF), and 42.4 mg peptide WL00 (SEQ ID NO: 1) was added to the solution. About 150 μl of triethylamine diluted 1:2 with DMF was added to the reaction mixture three times at 15 minute intervals, and then the solution was stirred for 17 hours at room temperature. The reaction solution was added to cold ether (300 ml), and the conjugate precipitates were filtered, washed with 200 ml ether, and dried. Amino acid analysis of the conjugate, PEG-WL00-OH, showed one mole of peptide WL00 incorporated per mole of PEG.

Doxorubicin (7.6 mg, Sigma) and PEG-WOO-OH (85 mg) were dissolved in 2 ml DMF and DCC solid (14 mg) was added to the solution. The reaction was carried out for 17 hours, precipitated with 200 ml ether, filtered, and washed with ether. The precipitate was dried under vacuum and then dissolved in PBS buffer. The solution was dialyzed for 25 hours with 3 changes of PBS buffer. Doxorubicin content of the product, PEG-WL00 (SEQ ID NO:1)-DXR, was determined by spectrophotometry at 490 nm.

### EXAMPLE 2

### A Control Composition Having the Formula PEG-Gly-Phe-Leu-Gly-ADR (hereinafter, “PEG-GFLG-ADR;” (GFLG is SEQ ID NO 1) was prepared according to the procedure of Example 1.

### EXAMPLE 3

### Preparation of Pendant 20 KD PEG-8PA-ONp

Purification of Starting Material: Innopeg 20M-8PA (15 g, Innophase Corporation) was dissolved in 100 ml of 5% water in methanol, and the solution introduced into a Spectra/Por MWCO 12-14,000 dialysis bag. The solution was dialyzed against two L of 5% aqueous methanol for 24 hours. The dialysate was replaced with fresh 5% aqueous methanol, and dialysis was continued for 24 hours. The process was repeated one additional time and the material in the bag concentrated to a thick syrup. The majority of the solvent was removed at 80° C. on the rotary evaporator using a vacuum pump and the glass recrystallized from 180 ml of acetone to yield 8.4 g of purified 20 KD pPEG-8PA. The combined dialysates after concentration in vacuo amounted to 5.6 g or 37.2% of the total. Before dialysis the polymer was determined to contain an average of 8.3 carboxyl groups per mole polymer by titration with 0.01N NaOH solution, while after dialysis the number was reduced to 6.5 carboxyl groups per mole.

### EXAMPLE 4

### Preparation of 20 KD mpPEG-8PA-GFLG-DXR

A conjugate of mpPEG (monomethyl pendant PEG), the GFLG spacer, and DXR (doxorubicin) was prepared.

Preparation of 20 KD mpPEG-8PA-ONp: Precautions should be taken to exclude water throughout this reaction sequence. A dry 500 ml one neck 24/40 round bottom flask was charged with 10.0 g (500 micromole, MW=20,000) of 20 KD mpPEG-8PA (Innophase) and 200 ml of dry toluene. Approximately 100 ml of toluene was distilled at atmospheric pressure while magnetically stirring to remove water. The stirring bar was removed and the balance of the toluene removed in vacuo. Under a dry argon atmosphere, dry dichloromethane (100 ml) was added to the syrup, followed by 973 mg (7000 micromoles) of p-nitrophenol and 81.1 mg (665 micromoles) of 4-dimethylaminopyridine (DMAP). Finely ground 1-[3-dimethylaminopropyl]-3-ethylcarbodiimide hydrochloride (EDC, 1340 mg, 7000 micromoles) was added in one portion while magnetically stirring the yellow solution. The reaction was allowed to proceed for two hours from the point at which all EDC had dissolved. Acetic acid (250 microliters) was added (to consume unreacted p-nitrophenol and EDC). The mixture was stirred for an additional 30 minutes, at which time 126.6 mg (665 micromoles) of p-toluenesulfonic acid monohydrate was added to neutralize the DMAP catalyst.

Stirring was continued until all of the solid had dissolved. Thereafter, the solution was concentrated in vacuo at 30 to 35° C. For purification 25 ml of methanol (MeOH) was added and the mixture stirred at room temperature until complete dissolution occurred. Isopropanol (IPA, 45 ml) was added, and the solution cooled in an ice bath until solid product appeared. The balance of 155 ml of IPA was added over a two to three minute period. The suspension was stirred for 30 minutes at 0° C., and filtered under an argon blanket. The filter plug was washed with 100 ml of 10% MeOH in IPA (v/v) at 0° C. in several portions, and dried to a damp solid. The flocculation procedure was repeated as described above except that the flask was mildly heated (a 37° C. water bath is sufficient) after addition of the methanol to cause dissolution of the damp solid. Cooling was initiated before adding any IPA. When a solid began to form, 200 ml of IPA was added over a two to three minute period. The isolated damp product was vacuum dried overnight to afford 10.16 g (98% yield) of 20 KD mpPEG-8PA-ONp as an off-white powder. No free p-nitrophenol, DMAP or p-nitrophenyl acetate could be detected in the product when it was analyzed by TLC (CH₃CN/sec-BuOH/toluene/AcOH/H₂O 1/1/1/1/1)]. The product was determined to contain 6.0 moles ONp/mole polymer (MW=20, 730) by determining the absorbance of a solution of ˜6 mg (weighed to the nearest 0.01 mg) in 50.0 ml 0.1N NaOH solution at 402.5 nm.

Preparation of 20 KD mpPEG-8PA-GFLG. 20 KD mpPEG-8PA-ONp (10 g, 482.4 micromoles) was introduced into a 500 ml one neck 24/40 round bottom flask, followed by six equivalents of the peptide GFLG (1136 mg, 2894 micromoles) and 30 ml of DMF while magnetically stirring the mixture under a dry argon blanket. After all solid had dissolved, 529 mg (4342 micromoles) of DMAP were added followed by 755 microliters (4342 micromoles) of diisopropylethyl amine (DIEA). The solution was stirred at room temperature for three hours. Concentrated ammonium hydroxide (750 microliters) was added with continued stirring for 90 minutes, followed by addition of 4.5 g p-toluenesulfonic acid. After complete dissolution of the solid, 30 ml of IPA were added and the stirred light yellow solution cooled in an ice bath until solid product began to form. The flask was removed from the ice bath, stirred at room temperature, and 170 ml of IPA added over a period of two to three minutes. The flask was again immersed in the ice bath, stirred for 30 minutes, and filtered under a blanket of argon. The filter cake was washed with 100 ml of 10% MeOH in IPA (v/v) at 0° C. in several portions and dried to a damp solid.

The solid was again flocculated. However, the mixture was heated in a 40° C. water bath after adding 25 ml of MeOH to cause dissolution. The flask was cooled in an ice bath while stirring the solution until solid began to form. The flask was then removed from the bath, and 200 ml of IPA was added over a two to three minute period with rapid stirring. The flask was again immersed in the ice bath, stirred for 30 minutes, and filtered under a blanket of argon. The filter cake washed with 100 ml of 10% MeOH in IPA (v/v) at 0° C. in several portions and dried to a damp solid. The isolated, off-white product was dried under high vacuum at room temperature overnight to yield 10.1 g of 20 KD mpPEG-8PA-GFLG (yield=92% based on PEG, 96.7% based on GFLG), which contained 5.8 moles GFLG/mole polymer (MW=22,160) by nitrogen analysis. Constant volume diafiltration can also be used to purify this intermediate. No contaminants, particularly p-toluenesulfonic acid, could be detected in the product when it was analyzed by TLC. No yellow color developed on treatment of a sample with 0.1N NaOH solution, indicating the absence of p-nitrophenol.

Preparation of 20 KD mpPEG-8PA-GFLG-DXR. 20 KD mpPEG-8PA-GFLG from above (9.89 g, 446.3 micromoles) was mixed with 5 equivalents of DXR HCl (Faith Eagle) (1294 mg, 2231.5 micromoles), 10 equivalents of p-nitrophenol (620.4 mg, 4463 micromoles), 2 equivalents of DMAP (108.9 mg, 892.6 micromoles), 10 equivalents of finely ground EDC (855.6 mg, 4463 micromoles) and 25 ml of dry DMF under an argon atmosphere. To the magnetically stirred mixture was added five equivalents of DIEA, (388 microliters, 2231.5 micromoles) and the reaction allowed to proceed overnight (16 hours). Water (500 microliters) was added to quench the reaction and stirring continued for two hours. Acetic acid (500 microliters) was added, and the solution stirred until homogeneous. The crude product was flocculated by adding 45 ml of IPA with cooling until solid appeared. IPA (150 ml) was added over a period of two to three minutes with stirring while continuing to cool the flask. The suspension was stirred for 30 minutes at 0° C. Thereafter, the product filtered under argon, washed with 50 ml of 10% MeOH in IPA (v/v, 0° C.) in several portions and the damp product dried under high vacuum.

The product was further purified by either chromatographing the crude product twice on LH-20 columns using methanol as eluent, or by constant volume diafiltration using 10% aqueous methanol. In either case, the purified product was isolated by concentrating the solution in vacuo, dissolving the product in 25 to 35 ml of methanol, and flocculating the product by the addition of 200 ml of IPA while cooling to 0° C. Filtration under argon followed by washing with 50 ml of 10% MeOH in IPA at 0° C. and high vacuum drying afforded 10 to 10.9 g of the final drug conjugate, which contained 2.5 to 3 moles of doxorubicin per mole of polymer. Free doxorubicin could be reduced to a level of 0.02% of the total doxorubicin depending on the method of purification. The product demonstrated excellent solubility in both PBS buffer and water.

### EXAMPLE 5

### Preparation of 20 KD mpPEG-8PA-WL00

20 KD mpPEG-8PA-ONp (Example 4) (933 mg, ˜42 μmoles, 44.2 mg ONp/g polymer) was introduced into a dry 50 mL round bottom flask. WL00 (150 mg, 297 μmoles) as the TFA salt (MW=507.46, 7.14 eq. compared to the ONp ester) was added. WL00=H-Gly-Phe-Leu-Gly-OH. DMF (3 mL) was added using a Pasteur pipette to rinse the sides of the flask free of solid. A magnetic stirring bar was introduced, and the flask stoppered. The mixture was heated to 40° C. to accelerate the dissolution of the solids, followed by stirring for 45 minutes until the bulk of the solid has dissolved. DIEA (104 μL, 594 μmoles, 2 eq. compared to WL00 content) was added. The reaction was stirred at room temperature for 2 h, followed by the addition of concentrated ammonium hydroxide (80 μL). Stirring was continued for 90 min before the addition of 250 mg p-toluenesulfonic acid monohydrate, with continued stirring until all p-toluenesulfonic acid dissolved. Subsequently, IPA (10 mL) was added, and the mixture was heated to 40° C.

The flask was cooled to room temperature overnight. Subsequently, the flask was placed in the refrigerator and cooled slowly to 3-4° C. The flask was removed from the refrigerator, immersed in an ice bath, followed by addition of IPA (10 mL) at 0° C. in one portion with stirring for 5 min. Stirring was ceased, and the solution was allowed to stand at 0° C. for 30 min. The white solid was filtered, and the filter cake washed with ice cold 10% MeOH/IPA (5 mL in three portions). The solid was washed with room temperature diethyl ether (5 mL total in three portions). The filter cake was dried under argon, and quantitatively transferred to a 10 mL Erlenmeyer flask followed by addition of 3 mL MeOH to dissolve the solid. IPA (10 mL) was added with heating to 40° C. The flask was allowed to cool to room temperature overnight. The flocculation procedure was repeated as above. Finally, the white to off-white solid was dried under vacuum overnight.

The synthesis yielded 781 mg (77%) of 20 KD mpPEG-8PA-WL00. Based upon nitrogen determination, the product contained 7 moles WL00/mole polymer, or 115 mg WL00g/polymer. No contaminants could be detected by TLC (CH₃CN/sec-BuOH/toluene/AcOH/H₂O 1/1/1/1/1/].

### EXAMPLE 6

### Preparation of 20 KD mpPEG-8PA-WL00(WL71)-DXR7

20 KD mpPEG-8PA-WL00-ONp (500 mg, 20.1 μmoles, 17.7 ONp) was prepared according to Example 5 and added to a dry vial. WL71 was added as the tris-TFA salt (MW=1515.4, 91 mg, 60.3 μmoles). WL71=H-Met-Val-Arg-Asn-Ile-Ser-Asn-Pro-Ser-Arg-OH, also identified as SEQ ID NO 9). Subsequently, DMF (1.5 mL) and DIEA (32 μL, 181 μmoles) were added, and the solution was stirred for 15 min. DXR HCl (47 mg, 80 μmoles) was then added to the vial, followed by continued stirring for 4 h. A second portion of DIEA (14 μL, 80.4 μmoles) was added followed by 50 μL of water to quench the reaction. Stirring was continued for 2 h, followed by addition of acetic acid (50 μL). For isolation, 5 mL IPA was added at 40° C., with stirring until a homogeneous solution was obtained. The solution was allowed to stand at room temperature in the dark overnight. The flask was then cooled to 0° C. in an ice bath, followed by addition of room temperature IPA (5 mL). The flask was chilled in the ice bath for 30 min.

The dark reddish-orange solid was filtered under argon into a 1-cm Buchner funnel attached to a 125 mL suction flask. The isolated drug conjugate was washed with 10% MeOH/IPA (5 mL total in three portions). The solid was washed with room temperature diethyl ether (5 mL in portions), then diafiltered from 10% aqueous MeOH (200 mL). Eight volumes of permeate were collected (1800 mL). The retentate was then concentrated, and the residue was dissolved in 1.5 mL MeOH, and flocculated as above. Finally, the product was dried under vacuum overnight (5×10⁻² torr).

The synthesis gave 520 mg (98% yield) of 20 KD mpPEG-8PA-WL00(WL71)-DXR, which contains 5.6% total DXR, 56.4 mg DXR/g polymer (by UV), 1.7% free DXR (95 mg free DXR/g polymer) by HPLC analysis, and 1.9 moles WL71/mole polymer (85 mg WL71/g polymer).

### EXAMPLE 7

### Preparation of 20 KD mpPEG-8PA-WL00(WL68-DXR

Preparation of 20 KD mpPEG-8PA-WL68.20 KD mpPEG-8PA-ONp (1.0 g, 44.7 μmoles) was introduced into a dry 50 ml RB flask followed by 6 eq. WL68 (492 mg, 268.2 μmoles), 1.33 eq. DMAP (7.3 mg, 59.45 μmoles) and 3 ml DMF. After the solids dissolved, 19 eq. DIEA (148 μL, 849.3 μmoles) was added and the reaction was stirred at RT overnight. Concentrated ammonium hydroxide (80 μL) was added and stirring was continued for 90 min followed by the addition of 1 ml acetic acid. After stirring 5 minutes, 6 ml IPA was added and the solution was placed in the fridge until solids began to form. Then 15 ml IPA was added over several minutes and the solution was cooled to 0° C. for 30 minutes. The suspension was filtered, washed with ice cold 10% MeOH/IPA and Et₂O, and dried to a damp solid. The solid was then dissolved in 3 ml MeOH (gently heated to afford dissolution) and the flocculation procedure was repeated. The solid was dried under vacuum overnight to give 1.26 g (95%) of 20 KD mpPEG-8PA-WL68. The product analyzed for 5.52 moles peptide/mole polymer (theoretical value is 6 moles peptide/mole polymer). No contaminants could be detected by TLC.

Preparation of 20 KD mpPEG-8PA-WL00-DXR. 20 KD mpPEG-8PA-WL68 (965 mg, 31.7 μmoles) (WL68 is SEQ ID NO 9) was added to a dry 50 ml RB followed by 6 eq. DXR HCl (110 mg, 190.2 μmoles), 12 eq. p-nitrophenol (53 mg, 380.4 μmoles), 12 eq. EDC HCL (73 mg, 380.4 μmoles), 1 eq. DMAP (4 mg, 31.7 μmoles) and 3 ml DMF. After dissolution, 6 eq. DIEA (34 μl, 190.2 μtmoles) was added and the solution was stirred overnight at room temperature. Water (100 μL) was added to quench the reaction, and stirring was continued for 2 h at which time acetic acid (100 μl) was added. For isolation, 10 ml IPA was added, followed by heating to 40° C., and cooling to room temperature for five hours before chilling in the fridge overnight. The chunks were broken up by adding 10 ml IPA at room temperature, and chilling in an ice bath for 30 min. Filter and wash with 10% MeOH/IPA and ether. The crude product was purified twice by LH-20 chromatography eluting with methanol (in 500 mg fractions). The fractions were concentrated and the residue was dissolved in 30 ml water and lyophilized overnight to afford 673 mg of 20 KD mpPEG-8PA-WL68-DXR as a fluffy red solid. The final drug conjugate was determined to contain 4.98% total DXR by UV and 2.7% free DXR by HPLC. Because of the high amount of free DXR, the conjugate was further purified by diafiltration (600 mg in 200 ml 90/10 MeOH/water and collected 10 volumes) to yield 600 mg which contains 5.2% total DXR and 1.06% free DXR.

### EXAMPLE 8

### Preparation of 20 KD mpPEG-8PA-WL00(WL75)-DXR

Preparation of 20 KD mpPEG-8PA-WL00-ONp. 20 KD mpPEG-8PA-WL00 (737 mg, 30.4 μmoles, 115 mg WL00/g polymer, ˜MW calc=24,240) was added to a dry 50 mL round bottom flask (24/40 one neck), followed by addition of dichloromethane (8 mL). A magnetic stirring bar was introduced, and stirring initiated. After dissolution, which required 30 min with intermittent heating to 35° C., p-nitrophenol (51 mg, 365 μmoles) was added. DMAP 97.8 mg, 64 μmoles) was added followed by addition of EDC (70 mg, 365 μmoles), with continued stirring. The solution was stirred for one hour following EDC dissolution, which required about 15 min. Acetic acid (100 μL) was subsequently added, and the mixture was stirred for an additional 30 mins.

p-Toluenesulfonic acid monohydrate (12.1 mg) was added to neutralize the DMAP catalyst, while continuing stirring. After the entire solid was dissolved, the stirring bar was removed, and the flask was transferred to a rotary evaporator. Approximately 5 mL of solvent was removed in vacuo on a rotary evaporator. The flask was raised from the water bath, and 20 mL of IPA was added. The flask was returned to the rotary evaporator, and an additional 3 mL of solvent was removed. The flask was raised from the water bath, and as the rotating flask cooled under vacuum, the product solidified. When the product has thoroughly solidified, the suspension was cooled to 0° C. for 1 h in an ice bath.

The white solid was filtered and recrystallized following similar procedures as in the preparation of 20 KD mpPEG-8PA-ONp (supra). The synthesis yielded 657 g (86% yield) of 20 KD mpPEG-8PA-WL00-ONp. No free p-nitrophenol, DMAP or p-nitrophenyl acetate can be detected by TLC (CHCl₃/MeOH/AcOH/H₂O) 50/7/7/1). The product was determined to contain 6.7 moles ONp/mole polymer (MW calc. ˜25,020) or 35.9 mg ONp/g polymer, by determining the absorbance of ˜5 mg in 50 mL 0.1N NaOH solution at 401.5 nm.

Preparation of 20 KD mpPEG-8PA-WL00(WL75)-DXR. 20 KD mpPEG-8PA-WL00-ONp (500 mg, 20 μmoles, 18 mg ONp) was added to a dry vial. WL75 as the bis-TFA salt (MW=574.4, 46 mg, 80 μmoles) was added. WL75=H-Arg-Gly-Asp-OH. (Arg-Gly-Asp is SEQ ID NO 10). Subsequently, DXR HCI (46 mg, 80 μmoles), DMF (1.5 mL) and DIEA (28 μL, 160 μmoles) were added, and the solution was stirred for four hours.

Water (50 μL) was added to quench the reaction. Stirring was continued for two hours, followed by addition of acetic acid (50 μL). For isolation, 1.5 mL DMF and 2.5 mL IPA were added at 40° C., with stirring until a homogeneous solution was obtained. The solution was cooled to room temperature with stirring, and the flask was then cooled to 16° C. Stirring was stopped once crystallization began. The flask was then removed from the water bath (10 min.), and left at room temperature overnight. IPA (7.5 mL) was then added with stirring, and the flask was cooled to 0° C. The flask was chilled in an ice bath for 30 min.

The dark reddish-orange solid was filtered under argon into 4.5 cm Buchner funnel attached to a 125 mL suction flask. The isolated drug conjugate was washed with 10% MeOH/IPA (5 ml total in three portions). The solid was washed with room temperature diethyl ether (5 mL in portions). The solid was dissolved in 3 mL MeOH, and 2.5 mL IPA was added at 40° C. The product was flocculated as previously described. Finally, the product was dried under high vacuum overnight (5×10−2 torr).

The synthesis yielded 370 mg (68%) of 20 KD mpPEG-8PA-WL00(WL75)-DXR which contains 7.3% DXR, 73 mg DXR/g polymer (by UV), 0.3% free DXR (0.2 mg free DXR/g polymer) by HPLC analysis, and 3.6 moles WL75/mole polymer (49.5 mg WL75/g polymer).

### EXAMPLE 9

### Preparation of 20 KD Methoxypolyethylene Glycol Acetate Containing Pendant Propionic Acid Groups

A magnetically stirred 1.55 g (77.78 μmoles) sample of purified mpPEG-8PA (MW=20,000, 8 pendant propionic acid moieties/mole polymer) in 30 ml of toluene was heated to just below the boiling point for 2 hours with 5.0 ml (5.4 g, 53 mmole) of acetic anhydride. The toluene was slowly distilled until 20 ml was collected and the balance of the toluene removed in vacuo. Water (10 ml) was added to the residue, and the milky solution was stirred overnight at room temperature to hydrolyze any acid anhydrides that were present. Methanol (40 ml) was added, the solution filtered, and diluted to a volume of 125 ml with 80/20 methanol/water. The solution was subjected to diafiltration (Pall Filtron Centramate cassette, 1 ft² surface area, MWCO=3 kD) using 80/20 methanol/water until 1 liter of permeate was collected. The retentate was withdrawn, the system flushed with methanol and the combined solutions removed in vacuo to yield 1.47 g. The product, which possessed a yellowish cast was dissolved in methanol, treated with 400 mg of Darco G-60 activated charcoal at the boiling point, then filtered. The filtrate was concentrated under vacuum. The residue was flocculated from 20 ml of acetone, with cooling to 0° C. to afford 1.19 g of off-white product after drying under high vacuum overnight. The product was found to contain 7.5 moles COOH/mole polymer, which compared favorably with a value of 7.6 moles COOH/mole polymer in the starting material. The SEC chromatogram showed an MP of 18,224 and a P_{d} of 1.56. The 500 MHz proton NMR spectrum shows the presence of the acetate group in the product as a singlet centered at 1.96 ppm. The product can be converted to the conjugates of the present invention, using various peptide components.

### EXAMPLE 10

### Preparation of 20 KD Methoxypolyethylene Glycol Hemisuccinate Containing Pendant Propionic Acid

A 5 g sample of purified methoxypolyethylene glycol (MW=20,000, 250 μmoles) containing an average of approximately eight pendant propionic acid groups and 0.5 g of succinic anhydride (5000 micromoles) dissolved in 50 ml of dry toluene was refluxed using a Dean Starke Trap for 2 hours. The toluene was removed in vacuo, and 1 ml of water plus 20 ml of acetone was added to the residue to hydrolyze excess succinic anhydride and any mixed anhydrides that formed during the preparation of the hemisuccinate. A reflux condenser was attached, the mixture stirred and heated 50 minutes at 50° C., cooled to room temperature and stirred for an additional 1.5 hours. Isopropanol (80 ml) was added to the solution and the flask was immersed in an ice bath. After one hour the white solid was filtered, washed with IPA at 0° C. and partially dried on the filter. The damp filter cake was re-flocculated from 100 ml of acetone to yield 4.08 g of product, and a third time from 80 ml of acetone to afford 3.5 g of 20 KD mpPEG-8PA-SA after drying under high vacuum overnight. Titration of the product with 0.01 N NaOH indicated the presence of 8.66 moles COOH/mole polymer compared to 7.64 mole COOH/mole polymer in the starting material, verifying the presence of the additional carboxyl group in the product. Size exclusion chromatography indicates an Mp of 22,550 with a P_{d} (M_{w}/Mₙ) of 1.58. The product can be converted to the conjugates of the present invention, using various peptide components.

### EXAMPLE 11

### Preparation of 20 KD Polyethylene Glycol Bis-Hemisuccinate Containing Pendant Propionic Acid Groups

A 5 g sample of polyethylene glycol (MW=22,000, 227 moles) purified by dialysis (regenerated cellulose, MWCO=12 to 14,000, against 95/5 water/methanol) containing an average of approximately eight pendant propionic acid groups and 1.0 g of succinic anhydride (10 millimoles) dissolved in 50 ml of dry toluene was refluxed using a Dean Starke Trap for 2 hours. The toluene was removed in vacuo and 10 ml of water was added to the residue to hydrolyze any anhydrides that were present. The mixture was stirred and heated for 15 minutes at 50° C., cooled to room temperature and stirred for an additional 1.5 hours. The solution was diluted with 20 ml of methanol, transferred to a dialysis bag (regenerated cellulose, MWCO=12 to 14,000), and dialyzed for 24 hours against 2 liters of 95/5 water/methanol (v/v). The contents of the bag were transferred to a round bottom flask and concentrated in vacuo. The residue was flocculated from a solution of the product in 10 ml of methanol by the addition of 100 ml of IPA at 0° C. Re-flocculation by the same procedure followed by high vacuum drying overnight afforded 5 g of pure white product. Titration of the product with 0.0 IN NaOH revealed the presence of 11.0 motes COOH/mole polymer compared to 9.5 motes COOH/mole polymer in starting material. This material can be used to prepare the conjugates of the present invention.

### EXAMPLE 12

### Preparation of 20 KD Polyethylene Glycol Diacetate Containing Pendant Propionic Acid Groups

A 10 g sample of commercial polyethylene glycol pendant propionic acid copolymer (MW=20,000, 500 micromoles) containing an average of 12.6 moles COOH/mole polymer in 40 ml of dry toluene containing 5.0 ml acetic anhydride (5.4 g, 53 mmole) was refluxed for 1.25 hours. The apparatus was reconfigured for distillation and 30 ml of liquid was distilled. The majority of volatile material was removed in vacuo, 20 ml of water added and the highly turbid mixture (some toluene was present) stiffed overnight at room temperature. The mixture was diluted with 180 ml of methanol, the clear solution filtered and the filtrate subjected to diafiltration as described above. The retentate was collected, the system purged with methanol and the combined solutions concentrated under vacuum. Isopropanol (IPA) was added to the residue and removed in vacuo to azeotrope water. The product was further purified by flocculation from 100 ml of acetone with cooling to 0° C. The isolated product, which possessed a yellow cast weighed 6.9 g after high vacuum drying to constant weight at 50° C. A 5 g sample was taken up in 50 ml of hot methanol, treated with 500 mg of Darco G-60 activated charcoal, and filtered. The filtrate was concentrated under vacuum, and the residue flocculated from 100 ml of acetone as described above. The white product weighted 4.44 g, and contained 9.75 moles COOH/mole polymer by titration. When the starting material specified above was subjected to the same purification procedures as described for the acetylated product, but without acetylation, the unacetylated material was found to contain 10.5 moles COOH/mole polymer. SEC results show an Mₚ of 20,655 and a P_{d} Of 1.45 (Sample 2, CA1043-179-1 following page). This product can be used to prepare conjugates of the present invention.

### EXAMPLE 13

### Preparation of 20 KD Polyethylene Glycol Diacetate Containing Pendant Propionic Acid Groups

A 25 g sample of commercial polyethylene glycol pendant propionic acid co-polymer (MW=20,000, 1.25 mmoles) containing an average of 12.6 moles COOH/mole polymer in 12.5 ml acetic anhydride (13.5 g, 132.5 mmoles) was stirred in an oil bath protected from atmospheric moisture at 115° C. for one hour. The majority of volatile material was removed in vacuo at 115° C. Subsequently, 25 ml of water added and the mixture was stirred for one hour at 50 to 60° C. The cooled mixture was diluted with 150 ml of methanol and filtered. The Buchner funnel was rinsed with a total of 75 ml of methanol in several portions, and the filtrate was subjected to diafiltration as in above examples using 90/10 methanol/water, except that 1.5 liters of permeate were collected. The retentate was collected, the system purged with methanol and the combined solutions concentrated were under vacuum. Isopropanol (IPA) was added to the residue and removed in vacuo to azeotrope water. The product was further purified by flocculation from 400 ml of acetone with cooling to 0° C. The isolated product after vacuum drying weighed 17 g and was shown to contain 10 moles COOH/mole polymer by titration. Starting material treated as in Example 4 above contained 10.5 moles COOH/mole polymer. SEC data show an Mₚ of 18,987, a P_{d} of 1.47. The 500 MHz proton NMR spectrum of the product shows the presence of the two acetate groups in the product at 2.02 ppm downfield from TMS. This product can be used to prepare conjugates of the present invention.

### EXAMPLE 14

### Preparation of 20 KD pPEG-10PA-2Ac-WL00-DXR

Preparation of 20 KD pPEG-10PA-2Ac-ONp. To a solution of 20 KD PPEG-1 OPA-2Ac (MW=22, 100, 500 mg, 22.62 micromoles), DMAP (3.67 mg, 30.08 micromoles) in 5 ml of dichloromethane was added, followed by addition of EDC (69 mg, 362 micromoles). The mixture was shaken for 3 hours at room temperature after dissolution of EDC. The reaction was quenched by the addition of 25 microliters of acetic acid, shaking continued for 30 minutes, and 5.95 mg p-toluenesulfonic acid (TsOH) was added to neutralize the DMAP. After dissolution of the TsOH, the solution was concentrated in vacuo at 35° C. The residue was flocculated from MeOH/IPA, then filtered after cooling to 0° C. and washing with MeOH/IPA. The damp filter cake was re-flocculated as above and dried under high vacuum overnight to yield 481 mg of 20 KD pPEG-10PA-2Ac-ONp. The product was determined to contain 8.6 moles ONp/mole polymer. No free p-nitrophenol or p-nitrophenyl acetate could be observed in the product when it was analyzed by TLC [CH₃CN/sec-BuOH/toluene/AcOH/H₂O 1/1/1/1/1].

Preparation of 20 KD pPEG-10PA-2Ae-WL00. A solution of the above product (481 mg, 20.78 micromole) in 2.0 ml of DMF was treated with 8.6 equivalents (90.5 mg, 178.69 micromoles) of WL00 TFA salt (WL00=GFLG) and 17.2 equivalents (62.5 microliters, 357.42 micromoles) of DIEA for 2 hours at room temperature. The reaction was quenched by the addition of 100 microliters of water. After stirring for two hours, 220 mg of TsOH was added, followed by 10 ml of IPA with cooling to 0° C. The flocculated product was filtered, washed with 10% MeOH/IPA and re-flocculated from MeOH/IPA. After high vacuum drying overnight, the product weighed 470 mg and was determined to contain 8.6 moles WL00/mole polymer by nitrogen analysis. No free WL00 could be detected in the product when it was analyzed by TLC and when the plate was developed with ninhydrin.

Preparation of 20 KD pPEG-10PA-2Ac-WL00-DXR. A 50 ml round bottom flask was charged with 434 mg 20 KD pPEG-10PA-2Ac-WL00 (17.28 micromole, 8.6 moles WL00/mole polymer), 100.2 mg DXR HCL (172.8 micromoles), 48.04 mg p-nitrophenol and 2.0 ml of DMF. The solution was magnetically stirred for 30 minutes and 66.25 mg of EDC added followed by 30 microliters of DIEA (172.8 micromoles). The reaction was stirred at room temperature for 20 hours, 100 microliters of water added and stirring continued an additional 2 hours. Finally, 100 microliters of AcOH was added. The product was isolated by diluting the solution with 10 ml of IPA, cooled to 0 C, filtered and washed with MeOH/IPA. The product was purified by passage through two 5×60 cm LH-20 columns eluted with MeOH. The product isolated weighed 450 mg, and contained 7.4% bound DXR and 1% free DXR, which corresponds to 3.7 moles DXR/mole polymer incorporated.

### EXAMPLE 15

### 20 KD mpPEG-8PA-WL68-DXR Drug Resistance Assay

The PC3 prostate carcinoma, as well as the MCF7-WT and SK-BR3 breast carcinoma cell lines were obtained from ATCC and maintained in RPMI 1640 (GibcoBRL, Grand Island, N.Y.) supplemented with 10% fetal bovine serum (FBS, Omega Scientific, Tarzana, Calif.), 100 IU/ml penicillin, 100 μg/ml streptomycin, and 2 mM L-glutamine (Irvine Scientific, Irvine, Calif.) (complete growth medium). Cells were harvested with 0.25% trypsin (GibcoBRL) after washing twice with phosphate-buffered saline (PBS, Irvine Scientific), then washed with complete medium, counted and checked for viability using trypan blue. Different passages of the cell line were tested for their resistance to WL68-DXR vs. native doxorubicin to evaluate the stability of WL68-DXR under storage at −70° C. The EDR assay for tumor drug resistance was performed as previously described (Kern et al., Nat. Cancer Inst. 82: 582-588 (1990); Fruehaul et al., PPO Updates 7: 1-16 (1993)). One HER-2-negative (MCF7-WT) and one HER-2-positive (SK-BR3) cell line were used in the study.

Tumor cells were suspended in 0.12% soft agar in complete medium, and plated at different cell concentrations (typically, 3,000 cells for the cell lines and 15,000 cells per well for fresh tumor specimens) onto a 0.4% agarose underlayer in 24-well plates. Plating cells on agarose underlayers supports the proliferation only of the transformed cells, ensuring that the growth signal stems from the malignant component of the tumor.

103 mg of 20 KD mpPEG-8PA-WL68-DXR (further referred to as WL-68-DXR) was dissolved in 10.3 ml of sterile PBS, aliquoted in plastic 1 ml tubes and kept at −70° C. until analysis. The doxorubicin equivalent for the WL68-DXR stock solution was 4.06 μg/ml. Native doxorubicin stock solution was prepared at 2.1 μg/ml. Stock solutions were diluted to 20× working solutions using the tissue culture medium, serially diluted and added to the 24-well plates. No significant changes in pH of the culture medium were observed under the above conditions. All experimental points were represented by two separate wells (duplicates). Positive controls were determined using at least 2 wells treated with an extremely high dose of cisplatin. Two wells containing tumor cells that were treated with PBS only served as negative controls for each 24-well plate in each experiment.

Cells were incubated with drugs under standard culture conditions for five days. Cultures were pulsed with tritiated thymidine (³H-TdR, New Life Science Products, Boston, Mass.) at 5 μCi per well for the last 48 hours of the culture period. Cell culture plates were then heated to 950° C. to liquefy the agarose, and cells were harvested onto glass fiber filters, which were then placed into counting vials containing liquid scintillation fluid. The radioactivity trapped on the filters was counted with a Beckman scintillation counter. The fraction of surviving cells was determined by comparing ³H-TdR incorporation in treated (experimental points) and untreated (negative control) wells.

Cell lines and fragments for fresh breast cancer specimens were fixed in 10% neutral buffered formalin for subsequent paraffin block preparation (See e.g., Mechetner et al., Clin. Cancer Res. 4: 389-398 (1998)). Four μm sections were cut from paraffin-embedded cell blocks, mounted on glass slides and heated overnight at 37° C. The slides were deparaffinized and rehydrated by consecutive submersions in Histoclear, absolute ethanol, 70% ethanol and distilled water. Tissue sections for HER-2 staining were pre-treated with citrate buffer and microwaving, as recommended by the HER-2 antibody manufacturer. IHC staining for HER-2 was performed on the Nexes (Ventana Medical Systems, Tucson, Ariz.) automated immunostainers according to the manufacturer's instructions. Sections were incubated in 3% hydrogen peroxide in distilled water for 10 minutes, and rinsed in tap water and distilled water. Before staining, each slide was subsequently incubated for 15 minutes at room temperature in 100 μl of blocking buffer (Protein Block, BioGenex) in a humid chamber. Excess of blocking buffer was shaken off, and the slides were incubated with primary mAbs at 37° C. (Nexes) and rinsed in PBS for 5 minutes.

A monoclonal antibody against HER-2 (clone 4E200) was purchased from Neomarkers, Inc. (Fremont, Calif.) and used at 1 μg/ml. Optimal working concentrations and incubation time for the HER-2 antibody have been determined in preliminary experiments. The sections were then incubated in peroxidase-labeled avidin/biotin secondary and tertiary reagents provided by the manufacturer of the Nexes immunostainer, washed in PBS and exposed to the peroxidase substrate solution (DAB). After immunoperoxidase staining, slides were rinsed with PBS for 5 minutes, counterstained with hematoxylin or methyl green (for image analysis) for 1 minute, rinsed for 10 minutes in tap water, dehydrated in ascending ethanol series, cleared in xylene, cover slipped in Permount and viewed under 40× magnification.

IHC staining for HER-2 was assessed by light microscopy. A minimum of 100 cells were analyzed for each section. Negative controls (irrelevant mouse Ig at matching concentrations) were run in parallel for all sections. The number of stained cells and their staining intensity were determined and presented as IHC score=(% of positive cells in the section)×(staining intensity +1).

### EXAMPLE 16

### Methods For Testing Her-2 Receptors

Monoclonal antibody (Mouse, IgG2b isotype, Transduction Laboratories, cat #E19420) against erbB2 (HER-2) receptors and Anti-mouse Ig ¹²⁵I labeled species-specific whole antibody (from sheep) (Amersham, cat#IM131) were used to test the exposition of HER-2 receptors on ovarian epithelial cancer cell line A2780/R. Primary antibody (against HER-2 receptor) was diluted by the binding buffer from the stock manufacture solution (250 μg/ml) to a concentration of 15 μg/ml. The A2870/R cells were cultured over night in fresh medium at a density of 0.5 million per wells in the 24 wells plate. Culture medium was washed out by PBS, and replaced with the binding buffer (50 mM PBS, 30 mM NaN₃, 0.5% BSA, pH 7.4). After keeping cells at 30° C. for 30 min, the binding buffer was replaced with the primary antibody solution I. Cells were incubated with antibody for 2 hours. Unbound antibody was washed three times with cold PBS. The secondary antibody (anti-mouse Ig antibody) was diluted tenfold and added to the cells (20 ul per wells). Cells were kept at 4° C. for one hour. Unbound secondary antibodies were washed with PBS. Cells were dissolved in IM NaOH and applied for counting ¹²⁵I-secondary antibody. To control the amount of the secondary antibody bound to the cell surface non-specifically, cells were incubated only with the secondary antibody without exposition to the primary antibody.

### EXAMPLE 17

### WL38 Binding To HER-2 Receptor

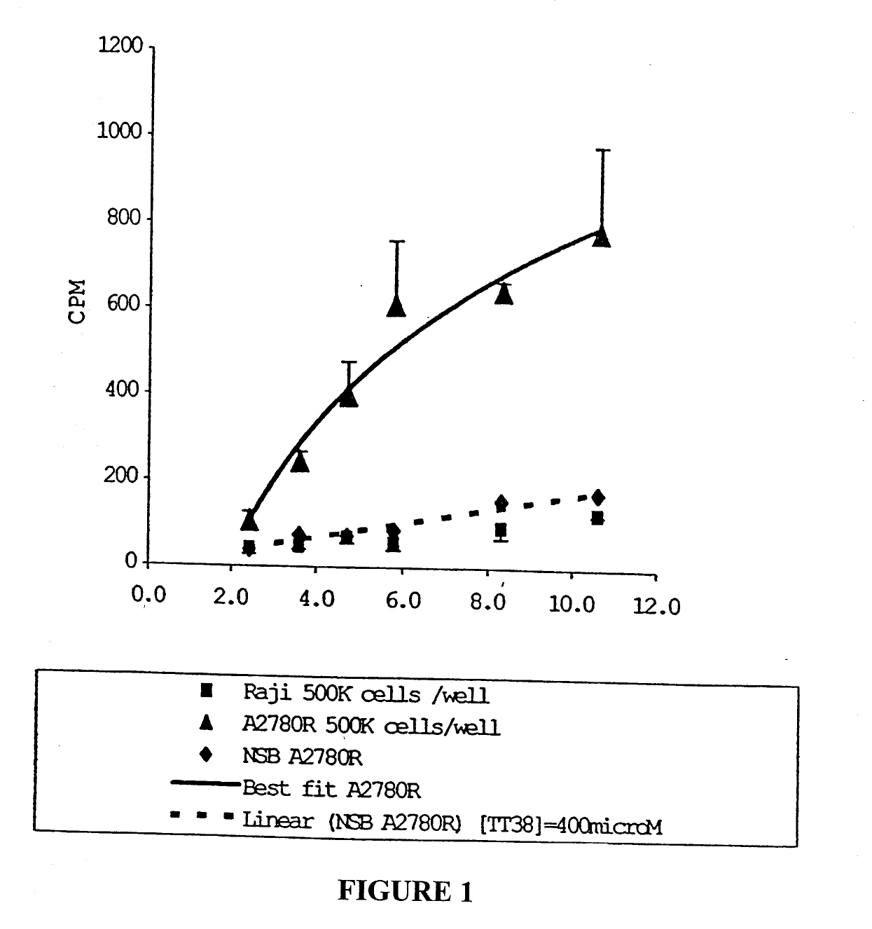

WL38 was iodinated by using lodo-Gen method described in herein. Unbound ¹²⁵I was removed by ion-exchange chromatography with MP-1 resin, (BIO-RAD AG). SK-Br3 cells were removed from the flask bottom by the cell scraper, suspended, and centrifuged at 500×g for 1 min. The cell pellet was suspended in the binding buffer (50 mM PBS, 30 mM NaN₃, 0.5% BSA, pH 7.4). The cell suspension (100 μl) containing 500,000 cells were placed into a glass tube and incubated with ¹²⁵I-WL38 peptides at various concentrations. Unbound peptide was removed by centrifugation. Cells were dissolved in 1M NaOH, and applied for ¹²⁵I counting. Binding of the WL38 peptide to Raji cell which were used as a negative control was performed in a similar manner. Non-specific binding of the WL38 to A2870/R cells was determined on the background of unlabeled WL3 8 at concentration 100 μM. The results are shown in FIG. 1.

### EXAMPLE 18

### Binding of PEG-TyrWL68-DXR Conjugates to Cell Surfaces

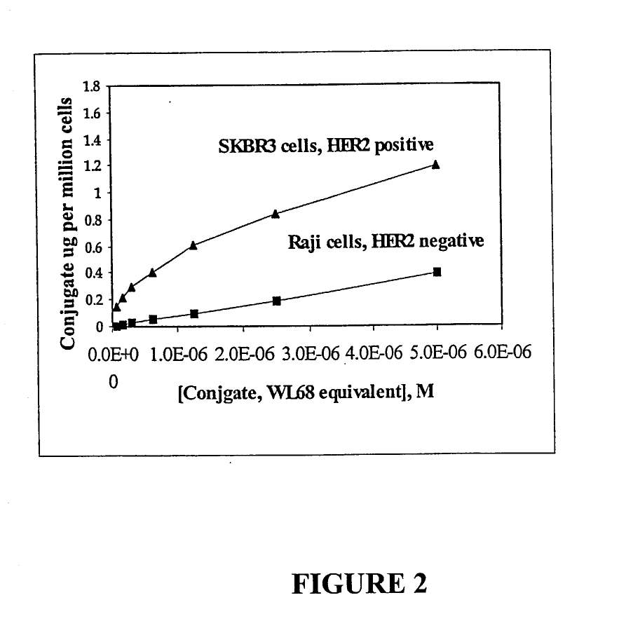

The PEG-TyrWL68 and PEG-TyrWL68-DXR (which comprise iodinated tyrosine conjugated to WL68) conjugates were iodinated by using the IODO-GEN method described herein. Unbound iodine was removed by SEC using PD 10 column. Concentration of labeled conjugate was determined by UV of DXR. SK-Br3 cells were scraped from the cell culture flask bottom, washed with the binding buffer and aliquoted into Ependorf centrifuge vials (450,000 cells per vial). Cells were centrifuged and suspended in 200 μl of the binding buffer. A mixture of ¹²⁵I-labeled and non-labeled conjugates at various concentrations were added to cell suspension, and incubated with cells for three hours. Unbound conjugates were washed out three times with PBS. At the final step of the washing, cells were transferred into new vials to avoid counting of conjugates which was not removed from the vial walls during washing procedure. Cells were dissolved in NaOH and applied for ¹²⁵I counting. Raji cells were used as a negative control of HER-2 binding to determine the amount of conjugates bound to the cell surface non-specifically. The results are shown in FIG. 2.

### EXAMPLE 19

### Procedure for In Vitro Cytotoxicity Assay

On the day prior to starting the assay, cells were fed with fresh tissue culture medium (RPMI 1640+10% FCS for most cell lines, use other media as appropriate). A conjugate test solution was prepared in culture medium, with a concentration that is twice the final desired test concentration. Typically with doxorubicin (DXR) conjugates, a DXR equivalent concentration range of 100 μM down to 0.1 μM, using 1:2 serial dilutions is used. All concentrations are calculated and reported in terms of final DXR concentration (as opposed to total conjugate concentration).

The cell suspension is prepared at a concentration of 1-2×10⁶ cells/Ml in culture medium. To each well of a 96-well, flat-bottom, microtiter plate (Falcon Microtest III assay plates, No. 3872) was added 50 μl of the cell suspension prepared above. A 50 μl of conjugate solution was added to each well of the assay plate. Duplicate wells should be prepared for each conjugate dilution tested. At this point, each well of the assay plate contains 5.0×10⁴ to 1.0×10⁵ cells, and conjugate at the final test concentration. The total volume is 100 μl per well.

The following blank and control wells were prepared: Plate Blank=100 μl of tissue culture medium only; cell control=50 μl cell suspension+50 μl tissue culture medium; and conjugate Blanks=50 μl 2× conjugate solution +50 μl tissue culture medium. The assay plate was incubated at 37° C. in a humidified, 5% Co₂ incubator for approximately two days. Following the incubation, 20 μl of MTS/PES solution (Promega CellTiter 96 AQ One Solution Cell Proliferation Assay, No. G-3580) was added to each well. The assay plate was incubated for an additional 2-4 hrs. Using a 96-well microplate reader, the absorbance of each well was measured at 490 nm. For each conjugate test concentration, the % cytotoxicity was calculated using the following formula: % Cytotoxicity=(1−[Abs. Conjugate Test Group/Abs. Cell Control])×100.

II. Results of Biological Assays of Doxorubicin Conjugates

The activity of doxorubicin conjugates was evaluated, including cytotoxicity studies, stability in human plasma, pharmacokinetics, body distribution, and enzymatic release as using the methods described above. The dose-response characteristics of the conjugates of the present invention were also tested.

A. Cytotoxicity Studies

In vitro cytotoxicity against human T-cell lymphoma cell line HUT 78 and human ovarian cancer cell lines A-2780R, as well as in vitro screening on several fresh human tumor specimens from different types of cancer were evaluated. The in vivo efficacy was determined using murine models for human cutaneous T-cell lymphoma. Free doxorubicin and liposomal doxorubicin (DOXIL®) were used as controls for the experiments. The composition for the mpPEG-WL00-DXR conjugates used in in vitro and in vivo studies is given in Table 1. The “WL00” moiety comprises the GFLG peptide (SEQ ID NO 1).

TABLE 1

| Lot Number | Percent DXR Loading of Conjugate |
|---|---|
| TM 1139-071 | 4 |
| TM 1192-026 | 8 |
| TM 1192-029-1 | 10 |
| TM 1192-038 | 10 |
| CA 1043-136 | 7 |

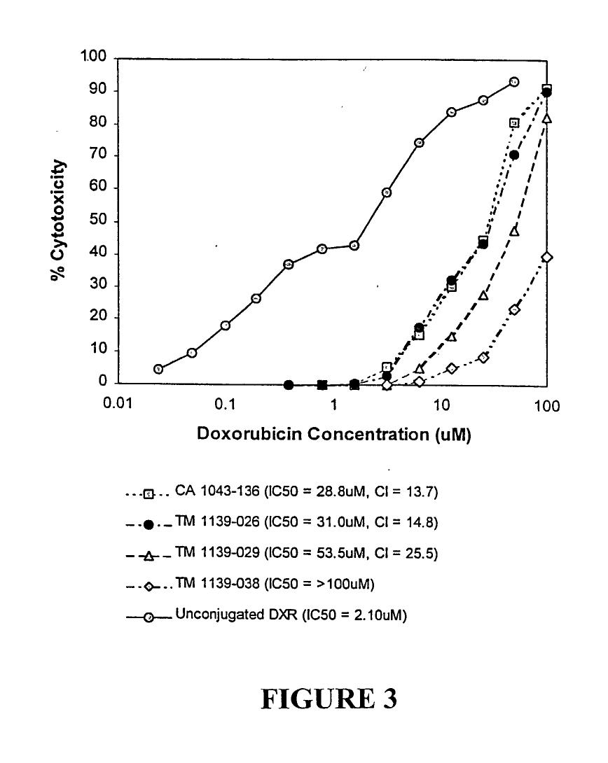

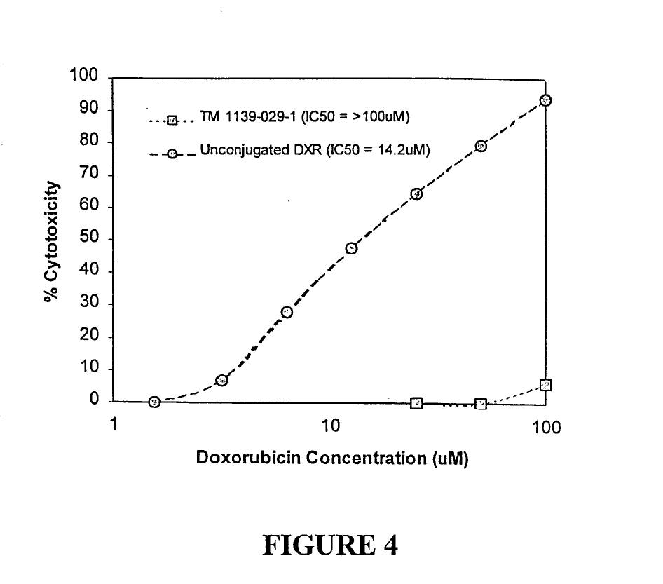

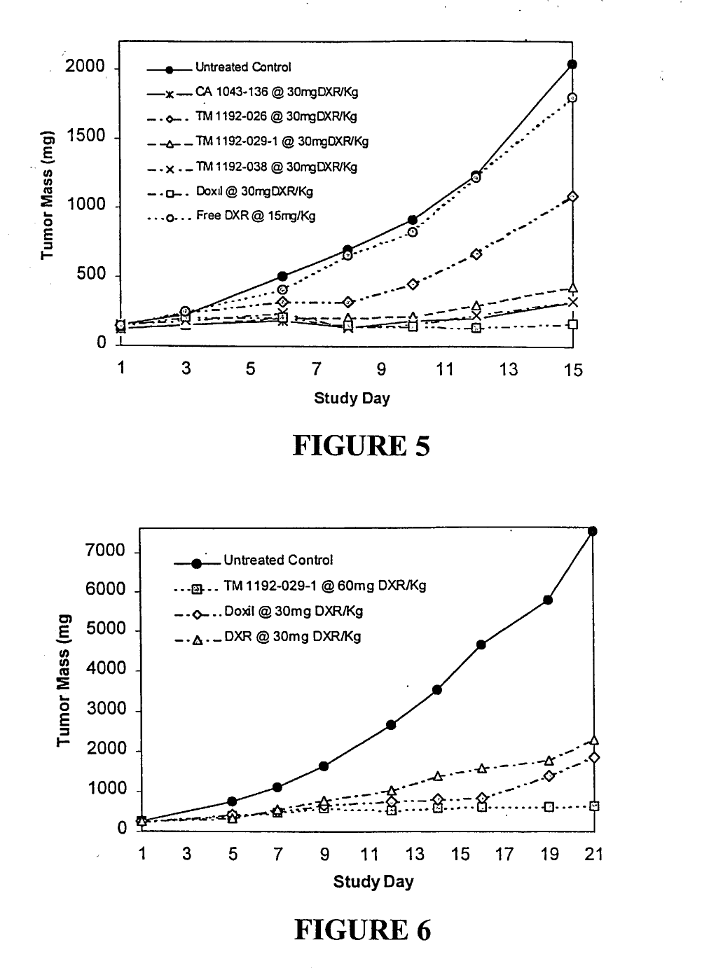

The in vitro cytotoxicity of mpPEG-WL00-DXR conjugate against the human cutaneous T-cell lymphoma line HUT 78 and human ovarian cancer cell line A-2780R is demonstrated in FIGS. 3 and 4, respectively. The IC₅₀ for unconjugated DXR against HuT 78 and A-27804 cell lines was found to be 2.10 μM and 14.2 μM, respectively, suggesting that these cell lines are resistant to doxorubicin. The conjugated doxorubicins labeled TM 1192-026 and CA 1043-136 showed similar cytotoxicity profile against HUT 78, with IC₅₀ value of 31.0 μM and 28.8 μM, respectively. The TM 1192-029-1 showed slightly higher value IC₅₀ of 53.5 μM against HUT 78. On the other hand, TM 1192-038 did not generate an IC₅₀ value against HUT 78, even at the highest DXR equivalent concentration of 100 μM, indicating lack of cytotoxicity of this conjugate against this cell line. Similarly, TM 1139-029-1 did not show cytotoxicity against A-2780R cell line.

The cytotoxicity testing of mpPEG-WL00-DXR and native doxorubicin ion fresh tumor specimens were also conducted. The results showed that the tumor from breast, colon, ovarian, lung, and non-hodgkins lymphoma patients are clearly very responsive to the conjugated doxorubicin. These results also suggested that mpPEG-WL00-DXR is effective against a wide range of tumors.

The efficacy of mpPEG-WL00-DXR in vivo was also evaluated. A single dose treatment of 30 mg/kg was used for the conjugate and DOXIL®, whereas 15 mg/kg (equivalent doxorubicin dose) was used for unconjugated DXR. Although the conjugate TM 1192-038 showed no cytotoxicity in vitro, it is as effective as TM 1192-029-1 and CA 1043-136. The percent tumor growth inhibition at day 15 for CA 1043-136, TM 1192-029-1 and TM 1192-038 was 84, 79 and 84, respectively. The tumor growth inhibition with DOXIL® was 92%, although DOXIL® also showed significant toxicity to the animal at 30 mg/kg. Treatment with unconjugated DXR at 15 mg/kg dose resulted in one death out of four animals tested, with a mean tumor growth inhibition of only 12% at day 15.

Efficacy of mpPEG-WL00-DXR conjugate was also compared with DOXIL® in a murine tumor model generated by subcutaneously implanting human ovarian cancer cell line A-2780R. At a single dose of DXR and DOXIL® at 30 mg/kg, three out of four animals died in DOXIL®-treated animals. In contrast, twice the dose of 60 mg/kg was used for the TM 1192-029-1, which resulted in one death out of four animals treated. At day 21, the tumor growth inhibition in the animal treated with TM 1192-029-1 animal was 92%. The unconjugated DXR at 30 mg/kg gave 70% tumor growth inhibition. These results indicate that conjugated DXR can be a more effective formulation than DOXIL® for the treatment of cancer.

In addition, a DXR conjugate with 8.7% DXR loading (TM 1192-107-1) was evaluated in both human T-lymphoma cell line HUT 78 and ovarian cancer cell line A-2780R animal tumor model. In the HUT 78 tumor study using 30 mg/kg DXR, five out of five animals died within seven days of the study period. In contrast, all the animals treated with 30 mg/kg of conjugated doxorubicins, CA 1043-136 and TM 1192-1071, survived until the end of the study period of 21 days. Both these conjugates showed more than 90% inhibition at the end of the study period. Furthermore, one out of five animals in both these groups did not have any observed tumor at the end of the study. It is also worth noting that the conjugate CA 1043-36 was very active after two years of storage as a lyophilized powder at 4° C.

B. Stability of PEG-WL00-DXR Conjugates in Human Plasma

The stability of PEG-WL00-DXR conjugates in human plasma was studied, using doxorubicin conjugates TM 1192-026 and TM 1192-029. Human plasma obtained from patients was used within two hours of collection. EDTA was used as the anticoagulant during the preparation of plasma. The conjugates were spiked with plasma at a concentration 50 μg/mL and incubated at 37° C. At various time points, samples were collected and frozen at −80° C.

The stability of PEG-WL00-DXR conjugate in plasma was evaluated by the appearance of free DXR and DXR metabolites in the plasma. An alternative method involving acid hydrolysis and determination of total DXR can also be used to validate the conjugate stability in plasma. DXR and its metabolites were extracted from plasma into an organic phase using procedures taken from Fraier et al. with slight modifications (i.e., using only one internal standard DNR; by adding 500 μL of 50 mM PBS to each sample; and by eliminating a terminal hexane rinse of the reconstituted sample). (See, Fraier et al., J. Pharm. Biomed. Anal. 22: 505-14 (2000); Fraier et al., J. Pharm. Biomed. Anal. 13: 625-633 (1995), incorporated herein by reference in their entirety). The amount of DXR and metabolites were determined by HPLC by fluorescence detection, using an excitation frequency of 485 nm and monitoring emission at 560 nm.

The amount of free DXR remained relatively unchanged throughout the duration of the incubation for both conjugates studied. After an initial small rise, the free DXR content gradually decreased and the concentration of metabolites steadily increased. The concentration of DXR and aglycone was determined from calibration curves run with known amounts of these chemicals as standard. The fluorescence response of aglycone was 1.5 fold higher than free DXR. The fluorescence responses of metabolites was in the range of one to two. The total value of DXR and its metabolites found in the plasma after fifty hours incubation was less than 10%, suggesting that the conjugate is stable in plasma.

C. Body Distribution of DXR

To study the ability of PEG-WL00-DXR conjugates to preferentially accumulate in tumor tissue, body distribution studies were performed using an mpPEG-WL00-DXR conjugate (TM 1192-029-1, see Table 1), free DXR and the liposomal formulation DOXIL®. NIH III mice bearing HuT 78 xenografts were administered with conjugated DXR and free DXR at a dose of 2.5 mg/kg of DXR equivalent. After 72 hours, the mice were sacrificed and tissue samples were prepared for analysis for free or total DXR.

The HPLC chromatogram of the organic phase extract of tissue homogenate of mice administered with the conjugate showed the presence of free DXR in all tissue analyzed. Trace amounts of aglycon was also observed. The total amount of DXR was determined after hydrolysis of free and conjugated DXR to generate aglycon. The amount of bound conjugate was computed as the difference between total and free DXR.

Distributions of free and bound DXR between various organs and tumor tissue of mice administered with TM 1192-029 conjugate. The amount of DXR was normalized per gram of tissue. The total amount of DXR per gram of the tumor tissue was significantly higher than the other organ or muscle, except in the spleen. With the exception of skeletal muscle, the heart had the lowest level of DXR accumulation of any tissue. The total DXR accumulated per gram of the tumor tissue is 5-6 fold higher, as compared to the heart or muscle tissue. Interestingly, the amount of free DXR released from the conjugate is very similar in various organs, except in the tumor and spleen.

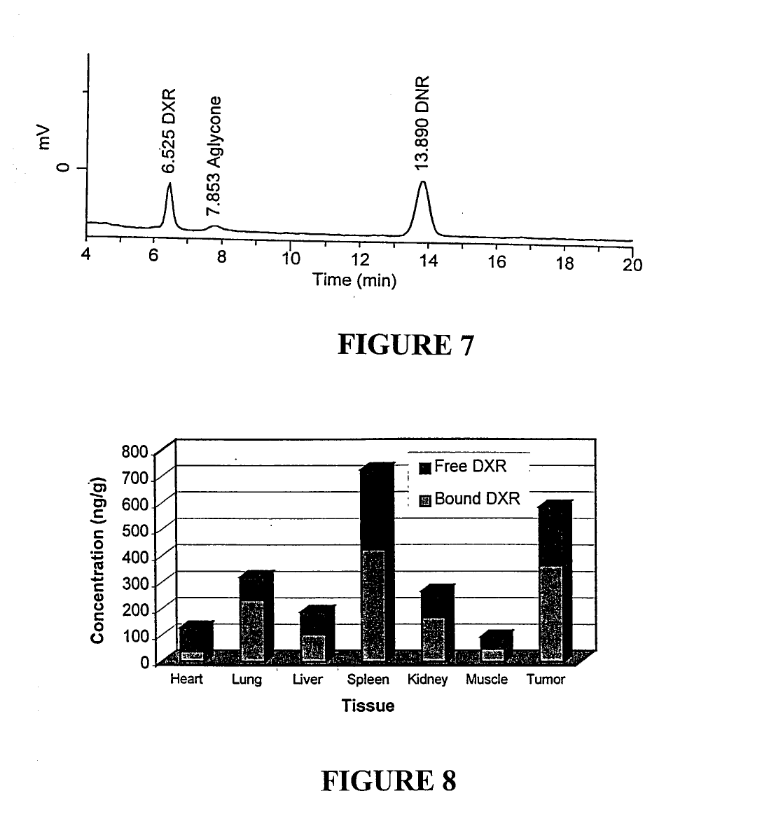

The HPLC chromatogram of the organic phase extract of tissue homogenate of mice administered with free DXR shows the presence of free DXR in all tissues analyzed. Trace amounts of aglycon was also observed. The DXR distribution is shown in FIG. 7, with the highest level of DXR per gram of tissue in the spleen. Approximately equal amounts of DXR were found per gram in the tumor, heart and muscle tissues.

The comparison of the body distribution of the conjugated DXR and free DXR reveals that with conjugated DXR, TM 1192-029, the tumor tissue contained nearly 14 times more DXR as compared to free DXR. See FIG. 8. These results demonstrate the targeting of conjugates to the tumor tissue.

D. Dose-Response Characteristics

The dose response characteristics of the conjugates of the present invention have been tested using 20 KD mpPEG-8PA-WL68-DXR on human breast carcinoma cell lines and fresh human breast carcinoma specimens.

Standard EPR Assay conditions (5 days of incubation, 1.5×10⁴ cells per well) were used for the tumor specimens. Two wide range of concentrations of 0.01 μg/ml to 1 μg/ml and 0.001 μg/ml to 0.2 μg/ml, were utilized for WL68-DXR and native doxorubicin on fresh breast carcinoma specimens, respectively. For the two cell lines, the range of concentrations for WL68-DXR and free doxorubicin were 0.01 μg/ml to 10 μg/ml and 0.001 μg/ml to 0.1 μg/ml, respectively.

To assess the stability of WL68-DXR under prolonged storage at −70° C., the cytotoxic activity of the drug was tested on PC3 human prostate carcinoma cell lines at two different time points (before and after freezing the stock solution). There was no significant difference in IC₅₀ values for either WL68-DXR (0.6 μg/ml before freezing the stock solution vs. 0.65 μg/ml after freezing) or native doxorubicin (0.027 μg/ml before freezing vs. 0.022 μg/ml after freezing) when both drugs were tested before freezing their stock solutions and after two weeks of storage at −70° C.

There was a statistically significant difference between the IC₅₀ values for WL68-DXR and native doxorubicin in the MCF7-WT (HER-2 negative) cell line, with values of 0.2±0.02 μg/ml and 0.013±0.004 μg/ml, respectively. There was also a statistical trend between the IC₅₀ values for WL68-DXR and native doxorubicin in the SK-BR3 (HER-2 positive) cell line, with values of 4.3±0.1 μg/ml and 0.03±0.02 μg/ml, respectively. Overall, the SK-BR3 cell line was more resistant to WL68-DXR and native doxorubicin than the MCF7-WT cell line.

In nine HER-2-negative breast carcinomas, IC₅₀ values for WL68-DXR varied between 0.07 μg/ml and 1 μg/ml (average: 0.49±0.35 μg/ml), while IC₅₀ values for native doxorubicin varied between and 0.004 μg/ml and 0.083 μg/ml (average: 0.025±0.025 μg/ml). The same trend was observed in the HER-2-positive group: IC₅₀ values for WL68-DXR (0.19±0.18 μg/ml) were significantly higher than those of native doxorubicin (0.019±0.020 μg/ml). Overall, there was a significant difference between IC₅₀ values for WL68-DXR and native doxorubicin.

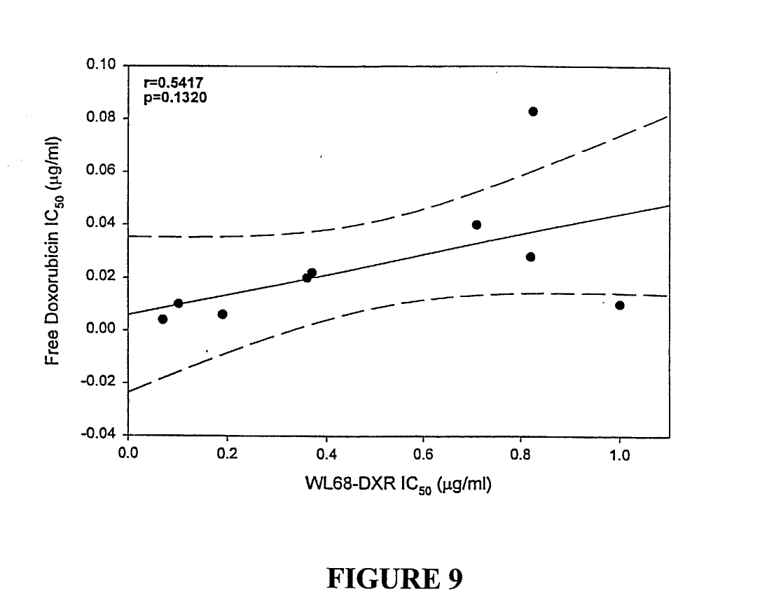

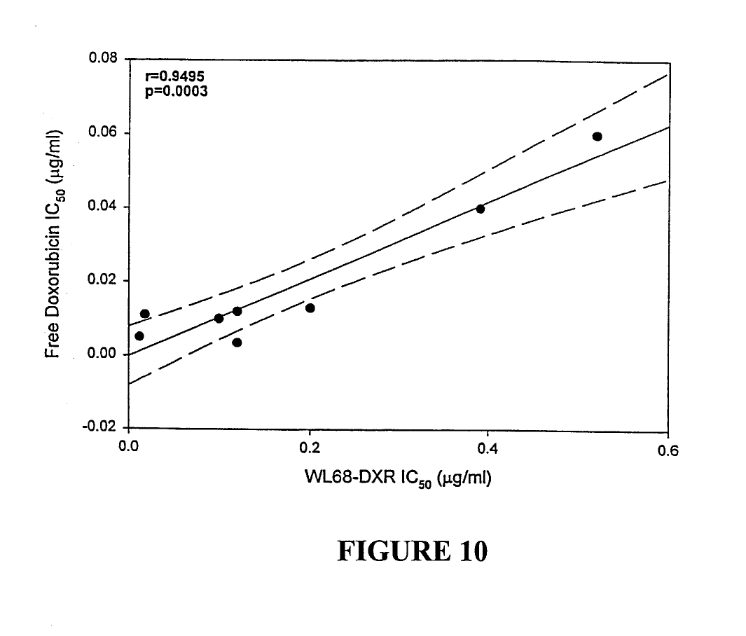

The results shown in FIGS. 9 and 10 demonstrate positive correlations between W168-DXR IC₅₀ and native doxorubicin IC₅₀ in the HER-2-negative (FIG. 9, r=0.5417, p=0.1320) and HER-2-positive groups (FIG. 10, r=0.9495, p=0.0003). In all breast carcinoma specimens, there was a strong statistically significant correlation (r=0.6115, p=0.0091) between IC₅₀ values for WL68-DXR and free doxorubicin. Although no statistically significant difference was found between native doxorubicin IC₅₀ values in the HER-2-negative and HER-2-positive groups (0.025±0.025 μg/ml and 0.019±0.020 μg/ml), WL68-DXR IC₅₀ values were significantly higher in the HER-2-negative group (0.49±0.35 μg/ml), as compared with the HER-2-positive group (0.19±0.18 μg/ml).

In contrast to these results, the HER-2-positive SK-BR3 cell line was more resistant to both doxorubicin drugs than the HER-2-negative MCF7-WT cell line. However, it is believed that data on drug resistance of primary tumors obtained from breast carcinoma patients (n=17) should reflect the in vivo cytotoxic/cytostatic effects of WL68-DXR and native doxorubicin more accurately than the data generated on the two breast carcinoma cell lines. This discrepancy may be explained by the low number of human breast carcinoma cell lines tested in the study (n=2), and/or major changes in tumor-associated gene expression that occur in tumor cell lines as a result of their prolonged cultivation in vitro.

III. Therapeutic Applications

The conjugates disclosed herein are useful in a wide variety of therapeutic applications, including but not limited to, enhancing the usefulness of cancer chemotherapeutic agents. The covalent binding of low molecular weight drugs to water-soluble polymer carriers permits enhancement of the specificity of drug action. Examples include compositions wherein a peptide ligand and a cytotoxic chemical agent, adriamycin or doxorubicin, are coupled to a branched PEG or a pendant PEG.

The compositions and conjugates according to the present invention can be used for targeted delivery of a chemical agent to target cells, generally by contacting the cells with the composition under conditions in which binding of or association with the ligand or target peptide to a receptor occurs. The chemical agent then acts on or within the targeted cell and the desired effect of the active agent can be defined to those cells having the receptor.

For example, a conjugate can be used as an effective anti-tumor agent in vivo for killing cancer cells and/or activated T cells. The conjugate also can be used for treating cancer and/or T-cell-associated diseases and tissue graft rejection. Such diseases include cancer, arthritis, cutaneous T-cell lymphoma, Hodgkin's disease, non-Hodgkin's lymphoma, skin cancers, psoriasis, graft rejection disease, multiple sclerosis, Type II diabetes mellitus, and disease resulting from HIV infection. The composition can be administered locally or systemically to a subject, such as a animal, e.g., a mammal, avian or fish. Preferably, the composition is administered to the subject by systemic administration, typically by subcutaneous, intramuscular, or intravenous injection, or intraperitoneal administration, which are methods well known in the art. Injectable preparations for such use can be made in conventional forms, either as a liquid solution, suspension, emulsion, or in a solid form suitable for preparation as a solution or suspension in a liquid prior to injection. Suitable excipients include, but are not limited to, water, saline, dextrose, glycerol, ethanol, and the like. If desired, minor amounts of auxiliary substances such as wetting or emulsifying agents, buffers, and the like may be added. Effective amounts of such compositions can be determined by those skilled in the art without undue experimentation according to the guidelines provided herein.

The above examples are presented to enable those skilled in the art to more clearly understand and practice the present invention. It is to be understood that preferred specific embodiments used to describe the invention are illustrative of the present invention, and are not intended to limit the scope of the invention. Other aspects of the invention will be apparent to those skilled in the art to which the invention pertains.

What is claimed is:

1. A conjugate consisting essentially of: a) a water-soluble biocompatible polymer; b) at least one targeting peptide directly linked to the polymer or indirectly linked to the polymer through a spacer peptide, and c) at least one molecule of a chemical agent releasably and directly coupled to the polymer or indirectly coupled to the polymer through a spacer peptide, wherein at least one of the targeting peptide and the spacer peptide comprises the sequence GFLG and/or GLFG.

2. The conjugate of claim 1, wherein the water-soluble biocompatible polymer is a polyalkylene oxide.

3. The conjugate of claim 2, wherein the polyalkylene oxide is polyethylene oxide.

4. The conjugate of claim 2, wherein said polyalkylene oxide is a member selected from the group consisting of alpha-substituted polyalkylene oxide derivatives, polyethylene glycol homopolymers and derivatives thereof, polypropylene glycol homopolymers and derivatives thereof, alkyl-capped polyethylene oxides, bis-polyethylene oxides, copolymers of poly(alkylene oxides), branched polyethylene glycols, star polyethylene glycols, pendant polyethylene glycols, block copolymers of poly(alkylene oxides) and activated derivatives thereof.

5. The conjugate of claim 2, wherein said polyalkylene oxide is an alkyl blocked pendant polyethylene glycol.

6. The conjugate of claim 5, wherein said pendant polyethylene glycol is a mono-methyl blocked pendant polyethylene glycol.

7. The conjugate of claim 1, comprising at least one spacer peptide directly linked to the polymer.

8. The conjugate of claim 7, wherein the spacer peptide is covalently, releasably coupled to the polymer.

9. The conjugate of claim 1, comprising two or more targeting peptides.

10. The conjugate of claim 1, wherein the chemical agent is selected from the group consisting of a cytotoxin, an immunosuppressant, a transforming nucleic acid, a gene regulator, a label, an antigen, and a drug.

11. The conjugate of claim 10, wherein the chemical agent is a cytotoxin selected from the group consisting of doxorubicin, taxol, cisplatin, methotrexate, cyclophosphamide, and a derivative of any thereof.

12. The conjugate of claim 1, wherein the targeting peptide specifically recognizes and binds a cell surface molecule expressed on a cancer cell.

13. The conjugate of claim 12, wherein the cell surface molecule is an integrin.

14. The conjugate of claim 7, wherein the spacer peptide comprises the peptide sequence GFLG and/or GLFG.

15. The conjugate of claim 1, further comprising a detectable label.

16. A composition comprising the conjugate of claim 1 and a carrier.

17. The composition of claim 16, wherein the carrier is a pharmaceutically acceptable carrier.

18. A method for screening a sample for a disease, comprising contacting a sample with an effective amount of the conjugate of claim 1 and determining whether a conjugate-receptor complex has formed.

19. The method of claim 18, further comprising the step of comparing the amount of complex formed in the sample with the amount of complex formed in a similarly treated control sample.

20. The method of claim 18, wherein said sample is selected from the group consisting of blood, tissue, saliva and urine.

21. A method of delivering a chemical agent to a subject, comprising administering to the subject an effective amount of the pharmaceutical composition of claim 17.

22. The conjugate of claim 1, wherein the at least one targeting peptide comprises the peptide sequence RGD (SEQ ID NO: 10).
